(19) United States
(12) Patent Application Publication
(10) Pub. No.: US 2007/0232526 A1
(43) Pub. Date: Oct. 4, 2007

(54) Bovine osteopontin formulations for the improvement of the wound healing process

(76) Inventors: Anne Staudt Kvistgaard, Viby J (DK); Esben Skipper Sorensen, Sabro (DK); Hans Burling, Viby J (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/639,297

(22) Filed: Dec. 15, 2006

Related U.S. Application Data
(60) Provisional application No. 60/750,797, filed on Dec. 16, 2005.

(51) Int. Cl. A61K 38/17

(52) U.S. Cl. 514/8

(57) ABSTRACT

Topical formulations containing bovine osteopontin for improved wound healing. The wound can be infected or inflamed or it can be a diabetic wound.

### TECHNICAL FIELD OF THE INVENTION

The invention concerns formulations of wound healing ointments, liquids, plasters and other devices containing bovine osteopontin in order to improve the wound healing process for wounds of every kind, including wounds difficult to heal, such as inflamed or infected wounds and especially wounds in diabetic patients.

### BACKGROUND OF THE INVENTION

It is known-that the macrophages, polymorph nuclear leukocytes, T-cells and other cells present in the lymph liquid associated with open wounds secrete osteopontin.

In mice it has been shown that osteopontin expression is up-regulated as early as 6 hours after wounding. Analyses of wound healing in mice lacking a functional osteopontin gene (knock-out mice) showed impaired wound healing ability. These wounds showed a significantly decreased level of debridement , a disorganization of matrix, and an alteration of collagen fibrillogonesis leading to smaller diameter collagen fibrils compared to wound in mice with a functional osteopontin gene.

Chitosan is being used as a wound-healing accelerator in veterinary medicine. The exact mechanism of action is not known. Though, chitosan has been shown to enhance the inflammatory actions of certain immune cells involved in wound healing and to induce the expression of osteopontin by those cells.

These findings prompted us to investigate the effect of administering a topical formulation containing osteopontin purified from bovine milk to different wound models.

### SUMMARY OF THE INVENTION

Surprisingly it has been found that bovine osteopontin can be used for improvement of wound healing process, whether the wounds are uninfected, infected or inflamed or from diabetic patients, without any allergic reactions across species.

### DETAILED DESCRIPTION OF THE INVENTION

The invention relates to a wound healing topical formulation comprising bovine osteopontin and an adjuvant.

Bovine osteopontin is osteopontin from milk and can be bought from Arla Foods Ingredients, Denmark or it can be obtained by passing acid whey through a strongly basic anionic resin aimed for chromatography like Q Sepharose from Amersham, UK. The osteopontin is eluted from the column using 1 M NaCl. The eluate is subsequently ultrafiltered and finally dried, e.g. using freeze-drying.

The adjuvant is a normal adjuvant for topical formulation, such as a carrier salve for an ointment.

The formulation can for example be an ointment, a liquid, a powder or a plaster. The adjuvant will be chosen from those normally used for such topical formulations. An example is carboxymethylcellulose for an ointment.

An ointment of the invention will preferably have an osteopontin level of 0.01% to 10%. Less than 0.01% will in most cases not give the wanted effect, and more than 10% will be superfluous. Preferred levels are 0.1% - 5%, 0.1- 2%, 0.1 to 1.5 or 0.5% to 1%. The amount can depend on the type of wound to be treated.

The invention also relates to the use of bovine osteopontin for the preparation of a topical drug for improved wound healing, including healing of inflamed wounds and wounds from diabetic patients.

The formulations can thus be used in a method for improved wound healing comprising administering to a patient in need thereof an effective amount of bovine osteopontin.

The patient can be a human or an animal.

In different wound models of mouse the effect of bovine osteopontin was investigated, namely a diabetes mice model with impaired wound healing ability, an infection compromised model and a healthy wild type model. It was found that the wound healing time was significantly reduced in all models by 1-3 days. However, the needed dose of bovine osteopontin varied with the used model. Lowest dose was needed for the wild mouse model and the highest for the infection mouse model. The ointment formulation was added varying amounts of bovine osteopontin, in the range 0.1-1.5%, for optimal performance depending on model.

Surprisingly we can thus state that bovine osteopontin could be used for treatment of wound healing improvement without allergic reactions across species.

Pharmaceutical Results

Three experimental test studies have been carried out using different levels of osteopontin. Bovine osteopontin was incorporated in a standard gel essentially consisting of carboxymethylcellulose. In the following tests figures which are written f. ex. as “0,27″ (as normal in Danish) should correctly have been f. ex. “0.27″ (as normal in English).

I. Accelerated wound healing in an impaired wound healing model

Introduction

Objective:

The purpose of the study was to test the pharmacological efficacy of Osteopontin on wound healing in a model of reduced wound healing in Type II diabetic mice.

Resume:

32 db/db mice (BKS.Cg-m+/+Lepr^{db}) were divided into 4 groups of 8. These mice developed diabetes type 2 and as a result had reduced wound healing.

Blood glucose was between 15-20 mM.

Additionally 8 wild type mice of the strain C57BLKS/j were used as controls of normal wound healing.

All mice were anesthetized, shaved and had a 8 mm wound made on their back with a 8 mm punch biopsy instrument at day 0.

From day 0-15 all animals were treated with carrier salve or 0.1%, 1% or 10% Osteopontin salve applied topical to the wound area.

From day 1-15 all animals were scored for wound appearance and had the wound diameter recorded.

On days 1, 4,10 and 15 the mice had their weight recorded.

During the whole experiment animals were pain treated with Rimaldyl as needed.

Results showed that Osteopontin treatment clearly worked better than no treatment at all. The Osteopontin 1.0% dose was superior to other Osteopontin doses in accelerating wound healing in diabetic mice.

The Osteopontin 0.1% dose was also quite successful in accelerating wound healing.

Justification:

The type II diabetic db/db mouse is known to have impaired wound healing and are as such an obvious strain for wound healing models.

Quality

The study was performed in accordance with Pipeline Biotech A/S, Standard Operating Procedure s (SOP's), unless otherwise stated.

Study timetable:

| | |
|---|---|
| Arrival of animals | 21.10.2004 |
| Start of treatment | 07.12.2004 |
| Live animal work complete | 22.12.2004 |

Test System

Test Article:

| | |
|---|---|
| Test article: | Osteopontin, topical application, 0.1%, 1% and 10% |
| Vehicle: | Carrier salve |

Species, Strain and Supplier

The study was performed in 32 DB mice (BKS.Cg-m+/+Lepr^{db}) and 8 C57BLKS/j (considered wildtype) from M&B-Taconic. Mice were 10-12 weeks upon arrival, but reached 17-19 weeks of age before start of the experiment due to a long waiting time for shipment of test articles.

Environment

The mice were single caged in standard Macrolon cages type 2. Bedding was filter paper.

Bedding was changed once a week in a laminar flow unit.

Temperature was 20° C. 24° C., and was controlled via the ambient ventilation system in the laboratory. Light cycle was 12-hour dark and 12-hour light (lights on 06.00).

Dietand Water

Diet was Harlan Teklad 2016 diet.

Water was UV-sterilized and water bottles were refilled when necessary during acclimatization and experiment.

Diet and water was administered ad libitum.

Animal Health -and Welfare

The animals had FELASA SPF-status and the housing and changing system was designed to assure that the SPF-status was preserved during the study. Educated personnel under veterinary supervision handled the animals. Daily records and decisions were made concerning animal welfare.

Pre-experimental procedures

Acclimatization and health procedures

Due to waiting time for shipment of test articles, animals were acclimatized for as long as 47 days.

Experimental procedures

Grouping

| Group | animals | strain | treatment |
|---|---|---|---|
| 1 | 1-8 | WT | Vehicle |
| 2 | 9-16 | DB | Vehicle |
| 3 | 17-24 | DB | Osteopontin 0.1% |
| 4 | 25-32 | DB | Osteopontin 1% |
| 5 | 33-40 | DB | Osteopontin 10% |

Analgesia

All mice were subjected to analgesia by administration of 5 mg/kg s.c. Rimaldyl 1 hour before wound procedures and once a day as needed during the experiment.

Introduction wounds

One hour after dosing of Rimadryl and approximately 15 minutes after hypnorm/dormicum anesthesia I wound was introduced on each mouse.

The wound was introduced on the dorsal skin of each mouse by the following. procedure:

1. The back of the mouse was shaven in a 2×3 cm area.

2. The 8 mm punch biopsy instrument was used to make a circular cut in the center area of the shaven area.

3. The skin piece was lifted a bit and carefully dissected free from the mouse.

4. Mice were returned to the cage

Treatment

All animals had either vehicle (carrier salve) or Osteopontin salve applied to the wound (0,05 mL) from day 0 (just after wound creation) to 14 (day before termination).

Application was performed just after scoring and measurement.

Observations and Measurements

No measurements were made the first day as the wounds needed to consolidate.

But wounds were scored and measured from day 1-15

The diameters of the wounds were measured longitudinally (on the line running from head to tail) and recorded each day.

The appearance of the wounds was scored and recorded each day. Weight was recorded on day 1, 4, 10 and 15.

Scoring of Wounds

Wounds were scored according to the following system:

0: Completely healed

1: Small wound still detected.

2: No scab but still wound

3: No original scab but new scab (old scab fallen off but new scab created)

4: original scab still on wound-.

Termination

At termination the mice were euthanized.

The wounds were dissected under aseptical conditions.

The wounds and approximately the area 0,5 cm surrounding the wound were placed in formalin buffer (Lilly's fluid) for further study as needed.

Sending Samples to Sponsor

Wound samples were retained until further notice from sponsor.

Study overview

| Day | Date | |
|---|---|---|
| −47 | 21.10.2004 | Animals arrived at Pipeline Biotech |
| 0 | 07.12.2004 | Introduction of wounds, treatment |
| 1 | 08.12.2004 | Weight, Measurement, score and treatment |
| 2 | 09.12.2004 | Measurement, score and treatment |
| 3 | 10.12.2004 | Measurement, score and treatment |
| 4 | 11.12.2004 | Weight, Measurement, score and treatment |
| 5 | 12.12.2004 | Measurement, score and treatment |
| 6 | 13.12.2004 | Measurement, score and treatment |
| 7 | 14.12.2004 | Measurement, score and treatment |
| 8 | 15.12.2004 | Measurement, score and treatment |
| 9 | 16.12.2004 | Measurement, score and treatment |
| 10 | 17.12.2004 | Weight, Measurement, score and treatment |
| 11 | 18.12.2004 | Measurement, score and treatment |
| 12 | 19.12.2004 | Measurement, score and treatment |
| 13 | 20.12.2004 | Measurement, score and treatment |
| 14 | 21.12.2004 | Measurement, score and treatment |
| 15 | 22.12.2004 | Weight, Measurement and score, termination with |
| | | preservation of skin area. |

Results

Average wound scores for each group in the study are summarized in the table below:

| Average wound scores | | | Scoring day | | | | | | | | | | | | | | |
|---|---|---|---|---|---|---|---|---|---|---|---|---|---|---|---|---|---|
| Group | Strain | Test Article | 1 | 2 | 3 | 4 | 5 | 6 | 7 | 8 | 9 | 10 | 11 | 12 | 13 | 14 | 15 |
| 1 | WT | Vehicle | 4.0 | 4.0 | 4.0 | 4.0 | 4.0 | 3.8 | 3.5 | 3.5 | 3.4 | 3.4 | 3.4 | 2.6 | 2.1 | 1.8 | 1.3 |
| 2 | DB | Vehicle | 4.0 | 4.0 | 4.0 | 4.0 | 4.0 | 4.0 | 4.0 | 4.0 | 3.7 | 3.7 | 3.1 | 2.7 | 1.9 | 0.6 | 0.4 |
| 3 | DB | Osteopontin | 4.0 | 4.0 | 4.0 | 4.0 | 4.0 | 4.0 | 4.0 | 3.8 | 3.5 | 3.5 | 2.7 | 2.5 | 0.5 | 0.0 | 0.0 |
| | | 0.1% | | | | | | | | | | | | | | | |
| 4 | DB | Osteopontin | 4.0 | 4.0 | 4.0 | 4.0 | 4.0 | 4.0 | 4.0 | 3.8 | 2.7 | 2.8 | 2.7 | 1.3 | 0.5 | 0.5 | 0.3 |
| | | 1% | | | | | | | | | | | | | | | |
| 5 | DB | Osteopontin | 4.0 | 4.0 | 4.0 | 4.0 | 4.0 | 4.0 | 4.0 | 3.9 | 3.3 | 3.4 | 2.9 | 2.6 | 1.1 | 0.9 | 0.7 |
| | | 10% | | | | | | | | | | | | | | | |

Scoring:
0: Completely healed
1: Small wound still detected.
2: No scab but still wound
3: No original scab but new scab (old scab fallen off but new scab created)
4: original scab still on wound.

These results are illustrated in the graph shown below:

[image]

Below are shown results of Mann-Whitney paired Rank-sum test (Wilcoxon two sample test) on Wound Scores. Results in control and test groups were compared to Group 2.

| | | | | | | Day | Day | Day | Day | Day | Day |
|---|---|---|---|---|---|---|---|---|---|---|---|
| Group | Day 6 | Day 7 | Day 8 | Day 8 | Day 9 | 10 | 11 | 12 | 13 | 14 | 15 |
| 1 | ns | ns | ns | ns | ns | ns | ns | ns | ns | ns | ns |
| 2 | — | — | — | — | — | — | — | — | — | — | — |
| 3 | ns | ns | ns | ns | ns | ns | ns | ns | ns | ns | ns |
| 4 | ns | ns | ns | ns | <0.01 | <0.01 | ns | ns | ns | ns | ns |
| 5 | ns | ns | ns | ns | ns | ns | ns | ns | ns | ns | ns |

n.s.: non significant

Conclusions:

| | | |
|---|---|---|
| Group 1: | WT; Vehicle | Not sign. different from Group 2. |
| Group 3: | DB; Osteopontin 0.1% | Not sign. different from Group 2. |
| Group 4: | DB; Osteopontin 1% | Significantly lower scores on Day 9 |
| | | and 10 than group 2. |
| Group 5: | DB; Osteopontin 10% | Not sign. different from Group 2. |

Average wound measurements

| | | | Measuring day (mm) | | | | | | | | | | | | | | |
|---|---|---|---|---|---|---|---|---|---|---|---|---|---|---|---|---|---|
| Group | Strain | Test Article | 1 | 2 | 3 | 4 | 5 | 6 | 7 | 8 | 9 | 10 | 11 | 12 | 13 | 14 | 15 |
| 1 | WT | Vehicle | 7.1 | 8.0 | 7.9 | 7.9 | 7.7 | 7.4 | 6.7 | 5.8 | 5.5 | 5.4 | 4.7 | 4.1 | 3.3 | 2.3 | 1.7 |
| 2 | DB | Vehicle | 5.9 | 6.5 | 6.6 | 6.5 | 6.4 | 6.1 | 5.8 | 6.2 | 5.7 | 5.6 | 5.5 | 4.6 | 2.7 | 1.3 | 1.0 |
| 3 | DB | Osteopontin | 5.0 | 5.4 | 5.6 | 6.2 | 6.0 | 5.9 | 5.8 | 5.5 | 4.9 | 4.7 | 3.2 | 3.2 | 0.5 | 0.0 | 0.0 |
| | | 0.1% | | | | | | | | | | | | | | | |
| 4 | DB | Osteopontin | 5.9 | 5.6 | 6.0 | 6.1 | 6.1 | 6.1 | 6.1 | 6.1 | 4.5 | 3.7 | 3.0 | 3.0 | 0.6 | 0.6 | 0.4 |
| | | 1% | | | | | | | | | | | | | | | |
| 5 | DB | Osteopontin | 6.3 | 6.5 | 7.9 | 7.7 | 7.5 | 7.6 | 7.5 | 6.8 | 6.6 | 6.4 | 5.5 | 4.6 | 2.2 | 1.6 | 1.5 |
| | | 10% | | | | | | | | | | | | | | | |

All wound measured on the line going from head to tail.
All wounds measured from the edge of the wound (between wound surface and normal skin).

These results are illustrated by the graph shown below:

[image]

Below are shown results of paired F- and Student t-tests on Wound Diameters. Results in control and test groups were compared to Group 2.

| | | | | | | | | | | Day | Day | Day | Day | Day | Day |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | 11 | 12 | 13 | 14 | 15 |
| 1 | — | ns | ns | ns | ns | ns | ns | ns | ns | ns | ns | ns | ns | ns | ns |
| 2 | — | — | — | — | — | — | — | — | — | — | — | — | — | — | — |
| 3 | — | ns | ns | ns | ns | ns | ns | <0.05 | ns | ns | <0.01 | <0.05 | <0.05 | <0.05 | <0.05 |
| 4 | — | ns | ns | ns | ns | ns | ns | ns | <0.05 | <0.01 | <0.01 | <0.05 | <0.05 | ns | ns |
| 5 | — | ns | <0.01 | <0.01 | <0.01 | <0.01 | <0.01 | ns | ns | ns | ns | ns | ns | ns | ns |

n.s.: non significant
Results of AUC (Area Under Curve) statistical analysis of Wound diameters are as follows: (Results in control and test groups were compared to Group 2.)
Group 1: <0.01
Group 3: <0.01
Group 4: <0.01
Group 5: <0.01

Conclusions:

| | | |
|---|---|---|
| Group 1: | WT; Vehicle | Generally sign. larger diameters than |
| | | Gr. 2 |
| Group 3: | DB; Osteopontin 0.1% | Generally sign. smaller diameters |
| | | than Gr. 2 |
| Group 4: | DB; Osteopontin 1% | Generally sign. smaller diameters |
| | | than Gr. 2 |
| Group 5: | DB; Osteopontin 10% | Generally sign. larger diameters than |
| | | Gr. 2 |

Wound areas:

Below are shown results of paired F- and Student t-tests on Wound Areas. Results in control and test groups were compared to Group 2.

| | | | | | | | | | | Day | Day | Day | Day | Day | Day |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | 11 | 12 | 13 | 14 | 15 |
| 1 | — | ns | ns | ns | ns | ns | ns | ns | ns | ns | ns | ns | ns | ns | ns |
| 2 | — | — | — | — | — | — | — | — | — | — | — | — | — | — | — |
| 3 | — | ns | ns | ns | ns | ns | ns | <0.05 | ns | ns | <0.01 | <0.05 | <0.05 | ns | ns |
| 4 | — | ns | ns | ns | ns | ns | ns | ns | <0.05 | <0.01 | <0.01 | <0.05 | <0.05 | ns | ns |
| 5 | — | ns | <0.01 | <0.01 | <0.01 | <0.01 | <0.01 | ns | ns | ns | ns | ns | ns | ns | ns |

n.s.: non significant
Results of AUC (Area Under Curve) statistical analysis of Wound Areas are as follows: (Results in control and test groups were compared to Group 2.)
Group 1: <0.05
Group 3: Non significant.
Group 4: Non significant.
Group 5: Non significant.

Conclusions:

| | | |
|---|---|---|
| Group 1: | WT; Vehicle | Generally sign. larger areas than |
| | | Gr. 2 |
| Group 3: | DB; Osteopontin 0.1% | Sign. smaller areas than Gr. 2 on |
| | | Days 8, 11, 12 and 13. |
| Group 4: | DB; Osteopontin 1% | Sign. smaller areas than Gr. 2 on |
| | | Days 9, 10, 11, 12 and 13. |
| Group 5: | DB; Osteopontin 10% | Sign. larger areas than Gr. 2 on |
| | | Days 3, 4, 5, 6 and 7. |

Wound Areas relative to Day 1:

Below are shown results of paired F- and Student t-tests on Wound arearelative to Day 1.

Results in control and test groups were compared to Group 2.

| | | | | | | | | | | Day | Day | Day | Day | Day | Day |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | 11 | 12 | 13 | 14 | 15 |
| 1 | — | ns | ns | ns | ns | ns | ns | <0.05 | <0.05 | ns | <0.05 | <0.05 | ns | ns | ns |
| 2 | — | — | — | — | — | — | — | — | — | — | — | — | — | — | — |
| 3 | — | ns | ns | ns | ns | ns | ns | ns | ns | ns | ns | ns | ns | ns | ns |
| 4 | — | <0.05 | ns | ns | ns | ns | ns | ns | <0.05 | <0.05 | <0.01 | <0.05 | ns | ns | ns |
| 5 | — | ns | ns | ns | ns | ns | <0.05 | ns | ns | ns | ns | ns | ns | ns | ns |

n.s.: non significant
Results of AUC (Area Under Curve) statistical analysis of Relative Wound Areas are as follows: (Results in control and test groups were compared to Group 2.)
Group 1: Non significant.
Group 3: Non significant.
Group 4: Non significant.
Group 5: Non significant.

Conclusions:

| | | |
|---|---|---|
| Group 1: | WT; Vehicle | Significantly larger wound areas than |
| | | Group 2 (DB, Vehicle) on day 8, 9, |
| | | 11 and 12. |
| Group 3: | DB; Osteopontin 0.1% | Does not deviate significantly from |
| | | Group 2. |
| Group 4: | DB; Osteopontin 1% | Significantly smaller wound areas |
| | | than Group 2 (DB, Vehicle) on day |
| | | 2, 9, 10, 11 and 12. |
| Group 5: | DB; Osteopontin 10% | Significantly larger wound areas than |
| | | Group 2 on day 7. |

Conclusion

The Osteopontin 1.0% salve was the most successful in accelerating wound healing in diabetic mice. A clear positive effect was seen on both wound scores and wound size. A positive effect was also observed on wound size in animals treated with 0.1% Osteopontin.

Archive

The final report as well as all raw data and results are -kept in the archives of Pipeline Biotech NS for a period of five (5) years from the end of the study.

TABLE 1
Wound scores

| 1 | | Scoring day | | | | | | | | | | | | | | |
|---|---|---|---|---|---|---|---|---|---|---|---|---|---|---|---|---|
| Animal | Strain | 1 | 2 | 3 | 4 | 5 | 6 | 7 | 8 | 9 | 10 | 11 | 12 | 13 | 14 | 15 |
| 1 | WT | 4 | 4 | 4 | 4 | 4 | 4 | 4 | 4 | 4 | 4 | 4 | 4 | 4 | 4 | 1 |
| 2 | WT | 4 | 4 | 4 | 4 | 4 | 4 | 4 | 4 | 4 | 4 | 4 | 4 | 4 | 4 | 4 |
| 3 | WT | 4 | 4 | 4 | 4 | 4 | 4 | 4 | 4 | 4 | 4 | 3 | 3 | 2 | 0 | 0 |
| 4 | WT | 4 | 4 | 4 | 4 | 4 | 4 | 4 | 4 | 4 | 4 | 4 | 3 | 3 | 3 | 3 |
| 5 | WT | 4 | 4 | 4 | 4 | 4 | 4 | 4 | 4 | 4 | 4 | 3 | 3 | 3 | 3 | 2 |
| 6 | WT | 4 | 4 | 4 | 4 | 4 | 2 | 2 | 2 | 2 | 2 | 3 | 1 | 0 | 0 | 0 |
| 7 | WT | 4 | 4 | 4 | 4 | 4 | 4 | 4 | 3 | 2 | 2 | 2 | 1 | 0 | 0 | 0 |
| 8 | WT | 4 | 4 | 4 | 4 | 4 | 4 | 2 | 3 | 3 | 3 | 2 | 2/3 | 1 | 0 | 0 |
| | Mean | 4.0 | 4.0 | 4.0 | 4.0 | 4.0 | 3.8 | 3.5 | 3.5 | 3.4 | 3.4 | 3.4 | 2.6 | 2.1 | 1.8 | 1.3 |
| | Std.dev. | 0.0 | 0.0 | 0.0 | 0.0 | 0.0 | 0.7 | 0.9 | 0.8 | 0.9 | 0.9 | 0.5 | 1.2 | 1.6 | 1.9 | 1.6 |
| 2 | | Scoring day | | | | | | | | | | | | | | |
| Animal | Strain | 1 | 2 | 3 | 4 | 5 | 6 | 7 | 8 | 9 | 10 | 11 | 12 | 13 | 14 | 15 |
| 9 | DB | 4 | 4 | 4 | 4 | 4 | 4 | 4 | 4 | 4 | 4 | 4 | 4 | 3 | 1 | 0 |
| 10 | DB | 4 | 4 | 4 | 4 | 4 | 4 | 4 | 4 | 4 | 4 | 4 | 2/3 | 2 | 1 | 1 |
| 11 | DB | 4 | 4 | (Dead) | — | — | — | — | — | — | — | — | — | — | — | — |
| 12 | DB | 4 | 4 | 4 | 4 | 4 | 4 | 4 | 4 | 4 | 4 | 4 | 4 | 3 | 1 | 1 |
| 13 | DB | 4 | 4 | 4 | 4 | 4 | 4 | 4 | 4 | 4 | 4 | 3 | 2 | 2 | 0 | 0 |
| 14 | DB | 4 | 4 | 4 | 4 | 4 | 4 | 4 | 4 | 4 | 4 | 4 | 3/1 | 0 | 0 | 0 |
| 15 | DB | 4 | 4 | 4 | 4 | 4 | 4 | 4 | 4 | 4 | 4 | 3 | 3 | 3 | 1 | 1 |
| 16 | DB | 4 | 4 | 4 | 4 | 4 | 4 | 4 | 4 | 2 | 2 | 0 | 1 | 0 | 0 | 0 |
| | Mean | 4.0 | 4.0 | 4.0 | 4.0 | 4.0 | 4.0 | 4.0 | 4.0 | 3.7 | 3.7 | 3.1 | 2.7 | 1.9 | 0.6 | 0.4 |
| | Std.dev. | 0.0 | 0.0 | 0.0 | 0.0 | 0.0 | 0.0 | 0.0 | 0.0 | 0.8 | 0.8 | 1.5 | 1.1 | 1.3 | 0.5 | 0.5 |
| 3 | | Scoring day | | | | | | | | | | | | | | |
| Animal | Strain | 1 | 2 | 3 | 4 | 5 | 6 | 7 | 8 | 9 | 10 | 11 | 12 | 13 | 14 | 15 |
| 17 | DB | 4 | 4 | 4 | 4 | 4 | 4 | 4 | 4 | 2 | 2 | 0 | 1 | 0 | 0 | 0 |
| 18 | DB | 4 | 4 | 4 | 4 | 4 | 4 | 4 | 4 | 4 | 4 | 3 | 3 | 0 | 0 | 0 |
| 19 | DB | 4 | 4 | (Dead) | — | — | — | — | — | — | — | — | — | — | — | — |
| 20 | DB | 4 | 4 | 4 | 4 | 4 | 4 | 4 | 4 | 4 | 4 | 4 | 4 | 3 | 0 | 0 |
| 21 | DB | 4 | 4 | 4 | 4 | 4 | 4 | 4 | 4 | 4 | 4 | 3 | 3 | 0 | 0 | 0 |
| 22 | DB | 4 | 4 | 4 | 4 | 4 | 4 | 4 | 4 | 4 | 4 | 3 | 2 | 0 | 0 | 0 |
| 23 | DB | 4 | 4 | 4 | 4 | 4 | 4 | 4 | 4 | 3 | 3 | 3 | 2 | 0 | 0 | 0 |
| 24 | DB | 4 | 4 | (Dead) | — | — | — | — | — | — | — | — | — | — | — | — |
| | Mean | 4.0 | 4.0 | 4.0 | 4.0 | 4.0 | 4.0 | 4.0 | 3.8 | 3.5 | 3.5 | 2.7 | 2.5 | 0.5 | 0.0 | 0.0 |
| | Std.dev. | 0.0 | 0.0 | 0.0 | 0.0 | 0.0 | 0.0 | 0.0 | 0.4 | 0.8 | 0.8 | 1.4 | 1.0 | 1.2 | 0.0 | 0.0 |
| 4 | | Scoring day | | | | | | | | | | | | | | |
| Animal | Strain | 1 | 2 | 3 | 4 | 5 | 6 | 7 | 8 | 9 | 10 | 11 | 12 | 13 | 14 | 15 |
| 25 | DB | 4 | 4 | 4 | 4 | 4 | 4 | 4 | 4 | 4 | 4 | 4 | 3 | 3 | 3 | 2 |
| 26 | DB | 4 | 4 | 4 | 4 | 4 | 4 | 4 | 4 | 2 | 2 | 0 | 0 | 0 | 0 | 0 |
| 27 | DB | 4 | 4 | 4 | 4 | 4 | 4 | 4 | 4 | 3 | 3 | 3 | 1 | 0 | 0 | 0 |
| 28 | DB | 4 | 4 | 4 | 4 | 4 | 4 | 4 | 4 | 2 | 2 | 3 | 1 | 0 | 0 | 0 |
| 29 | DB | 4 | 4 | 4 | 4 | 4 | 4 | 4 | 3 | 3 | 3 | 3 | 2/1 | 0 | 0 | 0 |
| 30 | DB | 4 | 4 | 4 | (Dead) | — | — | — | — | — | — | — | — | — | — | — |
| 31 | DB | 4 | 4 | 4 | 4 | 4 | 4 | 4 | 4 | 2 | 3 | 3 | 1 | 0 | 0 | 0 |
| 32 | DB | (Dead) | — | — | — | — | — | — | — | — | — | — | — | — | — | — |
| | Mean | 4.0 | 4.0 | 4.0 | 4.0 | 4.0 | 4.0 | 4.0 | 3.8 | 2.7 | 2.8 | 2.7 | 1.3 | 0.5 | 0.5 | 0.3 |
| | Std.dev. | 0.0 | 0.0 | 0.0 | 0.0 | 0.0 | 0.0 | 0.0 | 0.4 | 0.8 | 0.8 | 1.4 | 1.0 | 1.2 | 1.2 | 0.8 |
| 5 | | Scoring day | | | | | | | | | | | | | | |
| Animal | Strain | 1 | 2 | 3 | 4 | 5 | 6 | 7 | 8 | 9 | 10 | 11 | 12 | 13 | 14 | 15 |
| 33 | DB | 4 | 4 | 4 | 4 | 4 | 4 | 4 | 4 | 2 | 3 | 3 | 3 | 3 | 0 | 0 |
| 34 | DB | 4 | 4 | 4 | 4 | 4 | 4 | 4 | 3 | 3 | 3 | 3 | 3/1 | 0/1 | 0 | 0 |
| 35 | DB | 4 | 4 | 4 | 4 | 4 | 4 | 4 | 4 | 4 | 4 | 4 | 1 | 1 | 0 | 0 |
| 36 | DB | 4 | 4 | 4 | 4 | 4 | 4 | 4 | 4 | 4 | 4 | 2 | 3 | 0 | 0 | 0 |
| 37 | DB | 4 | 4 | 4 | 4 | 4 | 4 | 4 | 4 | 3 | 3 | 3 | 3 | 3 | 0 | 2 |
| 38 | DB | 4 | 4 | 4 | 4 | 4 | 4 | 4 | 4 | 4 | 4 | 2 | 3 | 0 | 0 | 0 |
| 39 | DB | 4 | 4 | (Dead) | — | — | — | — | — | — | — | — | — | — | — | — |
| 40 | DB | 4 | 4 | 4 | 4 | 4 | 4 | 4 | 4 | 3 | 3 | 3 | 3 | 3 | 0 | 3 |
| | Mean | 4.0 | 4.0 | 4.0 | 4.0 | 4.0 | 4.0 | 4.0 | 3.9 | 3.3 | 3.4 | 2.9 | 2.6 | 1.1 | 0.9 | 0.7 |
| | Std.dev. | 0.0 | 0.0 | 0.0 | 0.0 | 0.0 | 0.0 | 0.0 | 0.4 | 0.8 | 0.5 | 0.7 | 0.8 | 1.3 | 1.5 | 1.3 |

Scoring:
0: Completely healed
1: Small wound still detected.
2: No scab but still wound
3: No original scab but new scab (old scab fallen off but new scab created)
4: original scab still on wound.

TABLE 2
Wound measurements

| 1 | | Measuring day (mm) | | | | | | | | | | | | | | |
|---|---|---|---|---|---|---|---|---|---|---|---|---|---|---|---|---|
| Animal | Strain | 1 | 2 | 3 | 4 | 5 | 6 | 7 | 8 | 9 | 10 | 11 | 12 | 13 | 14 | 15 |
| 1 | WT | 8.08 | 8.04 | 7.20 | 7.36 | 7.15 | 7.22 | 5.77 | 5.91 | 6.67 | 6.44 | 6.42 | 5.79 | 5.46 | 5.71 | 1.99 |
| 2 | WT | 8.56 | 9.05 | 7.53 | 8.12 | 8.65 | 8.30 | 8.35 | 7.13 | 7.74 | 7.88 | 7.75 | 7.33 | 7.53 | 7.31 | 6.60 |
| 3 | WT | 6.00 | 7.32 | 7.49 | 7.95 | 7.03 | 7.84 | 7.37 | 6.06 | 5.92 | 5.77 | 3.80 | 2.68 | 3.35 | 0 | 0 |
| 4 | WT | 7.68 | 8.51 | 9.25 | 9.36 | 9.30 | 9.26 | 8.37 | 8.10 | 7.28 | 7.25 | 7.18 | 3.87 | 3.74 | 3.37 | 2.67 |
| 5 | WT | 9.28 | 10.45 | 10.45 | 10.11 | 10.00 | 9.94 | 8.63 | 7.94 | 7.76 | 8.47 | 4.39 | 4.14 | 4.03 | 2.35 | 2.36 |
| 6 | WT | 4.40 | 5.50 | 5.41 | 4.76 | 4.83 | 2.75 | 2.78 | 1.70 | 1.53 | 2.47 | 3.40 | 2.51 | 0 | 0 | 0 |
| 7 | WT | 5.77 | 5.92 | 6.77 | 6.59 | 5.86 | 5.31 | 6.30 | 4.12 | 2.63 | 1.60 | 1.44 | 2.52 | 0 | 0 | 0 |
| 8 | WT | 7.39 | 9.31 | 9.37 | 9.03 | 9.16 | 8.77 | 5.72 | 5.20 | 4.71 | 3.02 | 3.57 | 3.63 | 2.52 | 0 | 0 |
| | Mean | 7.1 | 8.0 | 7.9 | 7.9 | 7.7 | 7.4 | 6.7 | 5.8 | 5.5 | 5.4 | 4.7 | 4.1 | 3.3 | 2.3 | 1.7 |
| | Std.dev. | 1.6 | 1.7 | 1.6 | 1.7 | 1.8 | 2.4 | 2.0 | 2.1 | 2.4 | 2.6 | 2.2 | 1.7 | 2.6 | 2.9 | 2.3 |
| 2 | | Measuring day (mm) | | | | | | | | | | | | | | |
| Animal | Strain | 1 | 2 | 3 | 4 | 5 | 6 | 7 | 8 | 9 | 10 | 11 | 12 | 13 | 14 | 15 |
| 9 | DB | 6.87 | 7.17 | 7.78 | 6.61 | 6.14 | 6.33 | 5.52 | 5.86 | 5.93 | 5.60 | 5.41 | 5.12 | 3.58 | 1.37 | 0 |
| 10 | DB | 4.04 | 5.82 | 5.49 | 5.67 | 5.49 | 5.67 | 5.53 | 5.21 | 4.59 | 5.15 | 5.45 | 3.80 | 3.90 | 2.22 | 1.78 |
| 11 | DB | 4.16 | (Dead) | — | — | — | — | — | — | — | — | — | — | — | — | — |
| 12 | DB | 6.02 | 6.28 | 7.13 | 7.38 | 7.40 | 6.99 | 4.99 | 6.05 | 6.13 | 6.21 | 5.98 | 5.42 | 4.01 | 2.80 | 2.54 |
| 13 | DB | 5.34 | 6.08 | 6.16 | 6.55 | 7.04 | 6.57 | 6.49 | 6.46 | 6.43 | 6.43 | 5.18 | 2.93 | 2.95 | 0 | 0 |
| 14 | DB | 6.59 | 6.49 | 6.29 | 6.70 | 6.03 | 5.83 | 6.35 | 6.61 | 5.48 | 5.47 | 5.95 | 3.93 | 00 | 0 | 0 |
| 15 | DB | 6.29 | 5.49 | 6.06 | 6.25 | 6.19 | 5.99 | 6.13 | 7.12 | 7.03 | 6.54 | 4.74 | 5.56 | 4.61 | 2.41 | 2.39 |
| 16 | DB | 7.95 | 8.45 | 7.09 | 6.63 | 6.48 | 5.39 | 5.78 | 5.88 | 4.56 | 3.63 | | 5.28 | 0 | 0 | 0 |
| | Mean | 5.9 | 6.5 | 6.6 | 6.5 | 6.4 | 6.1 | 5.8 | 6.2 | 5.7 | 5.6 | 5.5 | 4.6 | 2.7 | 1.3 | 1.0 |
| | Std.dev. | 1.3 | 1.0 | 0.8 | 0.5 | 0.6 | 0.6 | 0.5 | 0.6 | 0.9 | 1.0 | 0.5 | 1.0 | 1.9 | 1.3 | 1.2 |
| 3 | | Measuring day (mm) | | | | | | | | | | | | | | |
| Animal | Strain | 1 | 2 | 3 | 4 | 5 | 6 | 7 | 8 | 9 | 10 | 11 | 12 | 13 | 14 | 15 |
| 17 | DB | 3.51 | 3.27 | 4.02 | 5.10 | 4.67 | 5.12 | 5.08 | 5.34 | 2.45 | 2.11 | — | 2.70 | 0 | 0 | 0 |
| 18 | DB | 5.41 | 7.27 | 5.66 | 6.05 | 5.61 | 5.81 | 5.53 | 6.25 | 6.77 | 6.21 | 3.31 | 2.67 | 0 | 0 | 0 |
| 19 | DB | 5.45 | (Dead) | — | — | — | — | — | — | — | — | — | — | — | — | — |
| 20 | DB | 4.32 | 6.14 | 6.50 | 6.17 | 6.18 | 5.89 | 5.83 | 5.06 | 5.41 | 5.38 | 4.85 | 5.99 | 2.84 | 0 | 0 |
| 21 | DB | 6.01 | 6.02 | 7.29 | 7.64 | 7.17 | 6.52 | 6.91 | 6.15 | 6.20 | 5.92 | 2.76 | 2.32 | 0 | 0 | 0 |
| 22 | DB | 4.40 | 4.20 | 4.64 | 5.85 | 6.11 | 6.10 | 5.59 | 5.79 | 4.92 | 4.79 | 2.28 | 2.93 | 0 | 0 | 0 |
| 23 | DB | 6.13 | 5.55 | 5.66 | 6.63 | 6.41 | 6.11 | 6.09 | 4.59 | 3.66 | 3.49 | 2.59 | 2.75 | 0 | 0 | 0 |
| 24 | DB | (Dead) | — | — | — | — | — | — | — | — | — | — | — | — | — | — |
| | Mean | 5.0 | 5.4 | 5.6 | 6.2 | 6.0 | 5.9 | 5.8 | 5.5 | 4.9 | 4.7 | 3.2 | 3.2 | 0.5 | 0.0 | 0.0 |
| | Std.dev. | 1.0 | 1.4 | 1.2 | 0.8 | 0.8 | 0.5 | 0.6 | 0.6 | 1.6 | 1.6 | 1.0 | 1.4 | 1.2 | 0.0 | 0.0 |
| 4 | | Measuring day (mm) | | | | | | | | | | | | | | |
| Animal | Strain | 1 | 2 | 3 | 4 | 5 | 6 | 7 | 8 | 9 | 10 | 11 | 12 | 13 | 14 | 15 |
| 25 | DB | 8.03 | 7.44 | 7.23 | 6.79 | 6.93 | 6.69 | 6.26 | 6.83 | 12.47 | 12.20 | 19.40 | 4.84 | 3.35 | 3.40 | 2.64 |
| 26 | DB | 3.96 | 3.63 | 4.03 | 5.00 | 5.43 | 5.36 | 4.80 | 4.13 | 2.99 | 2.52 | 0 | 0 | 0 | 0 | 0 |
| 27 | DB | 5.85 | 6.52 | 6.44 | 6.41 | 6.65 | 6.40 | 6.31 | 7.62 | 4.63 | 3.02 | 1.80 | 2.43 | 0 | 0 | 0 |
| 28 | DB | 5.08 | 4.56 | 5.78 | 5.43 | 5.53 | 5.53 | 6.06 | 5.87 | 4.75 | 3.73 | 3.41 | 4.50 | 0 | 0 | 0 |
| 29 | DB | 6.12 | 5.59 | 5.63 | 5.29 | 4.73 | 5.20 | 5.56 | 4.50 | 4.08 | 4.03 | 4.40 | 2.11 | 0 | 0 | 0 |
| 30 | DB | 4.47 | 4.96 | 5.65 | (Dead) | — | — | — | — | — | — | — | — | — | — | — |
| 31 | DB | 7.98 | 6.71 | 7.38 | 7.42 | 7.14 | 7.51 | 7.46 | 7.61 | 4.42 | 3.36 | 2.94 | 4.40 | 0 | 0 | 0 |
| 32 | DB | (Dead) | — | — | — | — | — | — | — | — | — | — | — | — | — | — |
| | Mean | 5.9 | 5.6 | 6.0 | 6.1 | 6.1 | 6.1 | 6.1 | 6.1 | 4.5 | 3.7 | 3.0 | 3.0 | 0.6 | 0.6 | 0.4 |
| | Std.dev. | 1.6 | 1.3 | 1.1 | 1.0 | 1.0 | 0.9 | 0.9 | 1.5 | 1.1 | 1.1 | 1.9 | 1.9 | 1.4 | 1.4 | 1.1 |
| 5 | | Measuring day (mm) | | | | | | | | | | | | | | |
| Animal | Strain | 1 | 2 | 3 | 4 | 5 | 6 | 7 | 8 | 9 | 10 | 11 | 12 | 13 | 14 | 15 |
| 33 | DB | 6.53 | 7.21 | 7.36 | 7.83 | 7.55 | 7.35 | 6.35 | 6.51 | 6.43 | 4.59 | 4.23 | 5.68 | 0 | 0 | 0 |
| 34 | DB | 5.81 | 6.03 | 7.09 | 7.58 | 7.27 | 7.35 | 7.06 | 3.56 | 3.22 | 3.69 | 3.43 | 3.13 | 2.16 | 0 | 0 |
| 35 | DB | 5.03 | 6.16 | 7.69 | 7.53 | 7.88 | 7.91 | 7.91 | 7.52 | 7.42 | 8.20 | 7.58 | 3.30 | 1.97 | 0 | 0 |
| 36 | DB | 6.05 | 6.13 | 7.46 | 7.39 | 7.40 | 7.20 | 7.30 | 7.21 | 7.81 | 7.56 | 3.46 | 2.28 | 0 | 0 | 0 |
| 37 | DB | 6.24 | 6.12 | 7.41 | 6.64 | 637 | 7.06 | 7.66 | 7.67 | 5.72 | 15.1 | 17.47 | 5.62 | 5.69 | 5.60 | 4.83 |
| 38 | DB | 6.33 | 6.85 | 8.40 | 7.67 | 7.40 | 7.70 | 7.59 | 7.52 | 8.10 | 8.00 | 6.13 | 5.08 | 0 | 0 | 0 |
| 39 | DB | 6.59 | (Dead) | — | — | — | — | — | — | — | — | — | — | — | — | — |
| 40 | DB | 7.49 | 6.99 | 9.60 | 9.05 | 8.96 | 8.79 | 8.70 | 7.68 | 7.34 | 7.21 | 8.06 | 6.81 | 5.77 | 5.89 | 5.68 |
| | Mean | 6.3 | 6.5 | 7.9 | 7.7 | 7.5 | 7.6 | 7.5 | 6.8 | 6.6 | 6.4 | 5.5 | 4.6 | 2.2 | 1.6 | 1.5 |
| | Std.dev. | 0.7 | 0.5 | 0.9 | 0.7 | 0.8 | 0.6 | 0.7 | 1.5 | 1.7 | 1.8 | 1.9 | 1.7 | 2.6 | 2.8 | 2.6 |

All wound measured on the line going from head to tail.
All wounds measured from the edge of the wound (between wound surface and normal skin).

TABLE 3
Wound areas

| 1 | | Measuring day (mm²) | | | | | | | | | | | | | | |
|---|---|---|---|---|---|---|---|---|---|---|---|---|---|---|---|---|
| Animal | Strain | 1 | 2 | 3 | 4 | 5 | 6 | 7 | 8 | 9 | 10 | 11 | 12 | 13 | 14 | 15 |
| 1 | WT | 51.25 | 50.74 | 40.69 | 42.52 | 40.13 | 40.92 | 26.13 | 27.42 | 34.92 | 32.56 | 32.35 | 26.32 | 23.40 | 25.59 | 3.11 |
| 2 | WT | 57.52 | 64.29 | 44.51 | 51.76 | 58.74 | 54.08 | 54.73 | 39.91 | 47.03 | 48.74 | 47.15 | 42.18 | 44.51 | 41.95 | 34.19 |
| 3 | WT | 28.26 | 42.06 | 44.04 | 49.61 | 38.80 | 48.25 | 42.64 | 28.83 | 27.51 | 26.13 | 11.34 | 5.64 | 8.81 | 0.00 | 0.00 |
| 4 | WT | 46.30 | 56.85 | 67.17 | 68.77 | 67.89 | 67.31 | 54.99 | 51.50 | 41.60 | 41.26 | 40.47 | 11.76 | 10.98 | 8.92 | 5.60 |
| 5 | WT | 67.60 | 85.72 | 85.72 | 80.24 | 78.50 | 77.56 | 58.46 | 49.49 | 47.27 | 56.32 | 15.13 | 13.45 | 12.75 | 4.34 | 4.37 |
| 6 | WT | 15.20 | 23.75 | 22.98 | 17.79 | 18.31 | 5.94 | 6.07 | 2.27 | 1.84 | 4.79 | 9.07 | 4.95 | 0.00 | 0.00 | 0.00 |
| 7 | WT | 26.13 | 27.51 | 35.98 | 34.09 | 26.96 | 22.13 | 31.16 | 13.32 | 5.43 | 2.01 | 1.63 | 4.99 | 0.00 | 0.00 | 0.00 |
| 8 | WT | 42.87 | 68.04 | 68.92 | 64.01 | 65.87 | 60.38 | 25.68 | 21.23 | 17.41 | 7.16 | 10.00 | 10.34 | 4.99 | 0.00 | 0.00 |
| | Mean | 40.1 | 50.4 | 49.4 | 49.1 | 47.1 | 43.3 | 34.8 | 26.1 | 24.0 | 22.6 | 17.7 | 12.9 | 8.7 | 4.3 | 2.3 |
| | Std.dev. | 17.6 | 20.9 | 20.7 | 20.0 | 21.4 | 23.7 | 18.4 | 17.2 | 18.0 | 21.0 | 16.7 | 13.0 | 14.8 | 15.6 | 11.6 |
| 2 | | Measuring day (mm²) | | | | | | | | | | | | | | |
| Animal | Strain | 1 | 2 | 3 | 4 | 5 | 6 | 7 | 8 | 9 | 10 | 11 | 12 | 13 | 14 | 15 |
| 9 | DB | 37.05 | 40.36 | 47.51 | 34.30 | 29.59 | 31.45 | 23.92 | 26.96 | 27.60 | 24.62 | 22.98 | 20.58 | 10.06 | 1.47 | 0.00 |
| 10 | DB | 12.81 | 26.59 | 23.66 | 25.24 | 23.66 | 25.24 | 24.01 | 21.31 | 16.54 | 20.82 | 23.32 | 11.34 | 11.94 | 3.87 | 2.49 |
| 11 | DB | 13.58 | (Dead) | — | — | — | — | — | — | — | — | — | — | — | — | — |
| 12 | DB | 28.45 | 30.96 | 39.91 | 42.75 | 42.99 | 38.36 | 19.55 | 28.73 | 29.50 | 30.27 | 28.07 | 23.06 | 12.62 | 6.15 | 5.06 |
| 13 | DB | 22.38 | 29.02 | 29.79 | 33.68 | 38.91 | 33.88 | 33.06 | 32.76 | 32.46 | 32.46 | 21.06 | 6.74 | 6.83 | 0.00 | 0.00 |
| 14 | DB | 34.09 | 33.06 | 31.06 | 35.24 | 28.54 | 26.68 | 31.65 | 34.30 | 23.57 | 23.49 | 27.79 | 12.12 | 0.00 | 0.00 | 0.00 |
| 15 | DB | 31.06 | 23.66 | 28.83 | 30.66 | 30.08 | 28.17 | 29.50 | 39.80 | 38.80 | 33.58 | 17.64 | 24.27 | 16.68 | 4.56 | 4.48 |
| 16 | DB | 49.61 | 56.05 | 39.46 | 34.51 | 32.96 | 22.81 | 26.23 | 27.14 | 16.32 | 10.34 | — | 21.88 | 0.00 | 0.00 | 0.00 |
| | Mean | 27.4 | 33.6 | 33.9 | 33.6 | 32.1 | 29.3 | 26.7 | 29.9 | 25.8 | 24.4 | 23.3 | 16.4 | 5.8 | 1.2 | 0.7 |
| | Std.dev. | 12.3 | 11.0 | 8.2 | 5.3 | 6.6 | 5.4 | 4.8 | 6.0 | 8.2 | 8.1 | 4.0 | 6.9 | 6.4 | 2.5 | 2.3 |
| 3 | | Measuring day (mm²) | | | | | | | | | | | | | | |
| Animal | Strain | 1 | 2 | 3 | 4 | 5 | 6 | 7 | 8 | 9 | 10 | 11 | 12 | 13 | 14 | 15 |
| 17 | DB | 9.67 | 8.39 | 12.69 | 20.42 | 17.12 | 20.58 | 20.26 | 22.38 | 4.71 | 3.49 | — | 5.72 | 0.00 | 0.00 | 0.00 |
| 18 | DB | 22.98 | 41.49 | 25.15 | 28.73 | 24.71 | 26.50 | 24.01 | 30.66 | 35.98 | 30.27 | 8.60 | 5.60 | 0.00 | 0.00 | 0.00 |
| 19 | DB | 23.32 | (Dead) | — | — | — | — | — | — | — | — | — | — | — | — | — |
| 20 | DB | 14.65 | 29.59 | 33.17 | 29.88 | 29.98 | 27.23 | 26.68 | 20.10 | 22.98 | 22.72 | 18.47 | 28.17 | 6.33 | 0.00 | 0.00 |
| 21 | DB | 28.35 | 28.45 | 41.72 | 45.82 | 40.36 | 33.37 | 37.48 | 29.69 | 30.18 | 27.51 | 5.98 | 4.23 | 0.00 | 0.00 | 0.00 |
| 22 | DB | 15.20 | 13.85 | 16.90 | 26.86 | 29.31 | 29.21 | 24.53 | 26.32 | 19.00 | 18.01 | 4.08 | 6.74 | 0.00 | 0.00 | 0.00 |
| 23 | DB | 29.50 | 24.18 | 25.15 | 34.51 | 32.25 | 29.31 | 29.11 | 16.54 | 10.52 | 9.56 | 5.27 | 5.94 | 0.00 | 0.00 | 0.00 |
| 24 | DB | (Dead) | — | — | — | — | — | — | — | — | — | — | — | — | — | — |
| | Mean | 19.9 | 23.0 | 24.9 | 30.6 | 28.5 | 27.6 | 26.8 | 24.0 | 18.9 | 17.0 | 7.8 | 8.2 | 0.2 | 0.0 | 0.0 |
| | Std.dev. | 7.5 | 11.9 | 10.6 | 8.6 | 7.7 | 4.2 | 5.9 | 5.6 | 11.8 | 10.4 | 5.8 | 9.2 | 2.6 | 0.0 | 0.0 |
| 4 | | Measuring day (mm²) | | | | | | | | | | | | | | |
| Animal | Strain | 1 | 2 | 3 | 4 | 5 | 6 | 7 | 8 | 9 | 10 | 11 | 12 | 13 | 14 | 15 |
| 25 | DB | 50.62 | 43.45 | 41.03 | 36.19 | 37.70 | 35.13 | 30.76 | 36.62 | 31.26 | 26.59 | 21.80 | 18.39 | 8.81 | 9.07 | 5.47 |
| 26 | DB | 12.31 | 10.34 | 12.75 | 19.63 | 23.15 | 22.55 | 18.09 | 13.39 | 7.02 | 4.99 | 0.00 | 0.00 | 0.00 | 0.00 | 0.00 |
| 27 | DB | 26.86 | 33.37 | 32.56 | 32.25 | 34.71 | 32.15 | 31.26 | 45.58 | 16.83 | 7.16 | 2.54 | 4.64 | 0.00 | 0.00 | 0.00 |
| 28 | DB | 20.26 | 16.32 | 26.23 | 23.15 | 24.01 | 24.01 | 28.83 | 27.05 | 17.71 | 10.92 | 9.13 | 15.90 | 0.00 | 0.00 | 0.00 |
| 29 | DB | 29.40 | 24.53 | 24.88 | 21.97 | 17.56 | 21.23 | 24.27 | 15.90 | 13.07 | 12.75 | 15.20 | 3.49 | 0.00 | 0.00 | 0.00 |
| 30 | DB | 15.69 | 19.31 | 25.06 | (Dead) | — | — | — | — | — | — | — | — | — | — | — |
| 31 | DB | 49.99 | 35.34 | 42.75 | 43.22 | 40.02 | 44.27 | 43.69 | 45.46 | 15.34 | 8.86 | 6.79 | 15.20 | 0.00 | 0.00 | 0.00 |
| 32 | DB | (Dead) | — | — | — | — | — | — | — | — | — | — | — | — | — | — |
| | Mean | 27.6 | 24.9 | 28.4 | 28.8 | 28.9 | 29.4 | 29.0 | 29.1 | 16.1 | 11.0 | 6.9 | 7.3 | 0.2 | 0.3 | 0.2 |
| | Std.dev | 15.5 | 11.8 | 10.4 | 9.3 | 9.1 | 9.0 | 8.5 | 14.2 | 8.0 | 7.7 | 8.1 | 7.8 | 3.6 | 3.7 | 2.2 |
| 5 | | Measuring day (mm²) | | | | | | | | | | | | | | |
| Animal | Strain | 1 | 2 | 3 | 4 | 5 | 6 | 7 | 8 | 9 | 10 | 11 | 12 | 13 | 14 | 15 |
| 33 | DB | 33.47 | 40.81 | 42.52 | 48.13 | 44.75 | 42.41 | 31.65 | 33.27 | 32.46 | 16.54 | 14.05 | 25.33 | 0.00 | 0.00 | 0.00 |
| 34 | DB | 26.50 | 28.54 | 39.46 | 45.10 | 41.49 | 42.41 | 39.13 | 9.95 | 8.14 | 10.69 | 9.24 | 7.69 | 3.66 | 0.00 | 0.00 |
| 35 | DB | 19.86 | 29.79 | 46.42 | 44.51 | 48.74 | 49.12 | 49.12 | 44.39 | 43.22 | 52.78 | 45.10 | 8.55 | 3.05 | 0.00 | 0.00 |
| 36 | DB | 28.73 | 29.50 | 43.69 | 42.87 | 42.99 | 40.69 | 41.83 | 40.81 | 47.88 | 44.87 | 9.40 | 4.08 | 0.00 | 0.00 | 0.00 |
| 37 | DB | 30.57 | 29.40 | 43.10 | 34.61 | 31.85 | 39.13 | 46.06 | 46.18 | 25.68 | 25.50 | 25.06 | 24.79 | 25.42 | 24.62 | 18.31 |
| 38 | DB | 31.45 | 36.83 | 55.39 | 46.18 | 42.99 | 46.54 | 45.22 | 44.39 | 51.50 | 50.24 | 29.50 | 20.26 | 0.00 | 0.00 | 0.00 |
| 39 | DB | 34.09 | (Dead) | — | — | — | — | — | — | — | — | — | — | — | — | — |
| 40 | DB | 44.04 | 38.36 | 72.35 | 64.29 | 63.02 | 60.65 | 59.42 | 46.30 | 42.29 | 40.81 | 51.00 | 36.41 | 26.13 | 27.23 | 25.33 |
| | Mean | 30.7 | 33.2 | 48.5 | 46.2 | 44.7 | 45.6 | 44.3 | 36.4 | 34.0 | 32.4 | 23.8 | 16.3 | 3.9 | 2.1 | 1.8 |
| | Std.dev. | 6.9 | 5.1 | 11.5 | 8.9 | 9.4 | 7.4 | 8.6 | 13.1 | 15.1 | 16.8 | 16.8 | 11.8 | 12.0 | 12.7 | 10.8 |

All wound measured on the line going from head to tail.
All wounds measured from the edge of the wound (between wound surface and normal skin).

TABLE 4
Wound areas relative to Day 1

| 1 | | Measuring day (Wound areas relative to Day 1) | | | | | | | | | | | | | | |
|---|---|---|---|---|---|---|---|---|---|---|---|---|---|---|---|---|
| Animal | Strain | 1 | 2 | 3 | 4 | 5 | 6 | 7 | 8 | 9 | 10 | 11 | 12 | 13 | 14 | 15 |
| 1 | WT | 1.00 | 0.99 | 0.79 | 0.83 | 0.78 | 0.80 | 0.51 | 0.53 | 0.68 | 0.64 | 0.63 | 0.51 | 0.46 | 0.50 | 0.06 |
| 2 | WT | 1.00 | 1.12 | 0.77 | 0.90 | 1.02 | 0.94 | 0.95 | 0.69 | 0.82 | 0.85 | 0.82 | 0.73 | 0.77 | 0.73 | 0.59 |
| 3 | WT | 1.00 | 1.49 | 1.56 | 1.76 | 1.37 | 1.71 | 1.51 | 1.02 | 0.97 | 0.92 | 0.40 | 0.20 | 0.31 | 0.00 | 0.00 |
| 4 | WT | 1.00 | 1.23 | 1.45 | 1.49 | 1.47 | 1.45 | 1.19 | 1.11 | 0.90 | 0.89 | 0.87 | 0.25 | 0.24 | 0.19 | 0.12 |
| 5 | WT | 1.00 | 1.27 | 1.27 | 1.19 | 1.16 | 1.15 | 0.86 | 0.73 | 0.70 | 0.83 | 0.22 | 0.20 | 0.19 | 0.06 | 0.06 |
| 6 | WT | 1.00 | 1.56 | 1.51 | 1.17 | 1.21 | 0.39 | 0.40 | 0.15 | 0.12 | 0.32 | 0.60 | 0.33 | 0.00 | 0.00 | 0.00 |
| 7 | WT | 1.00 | 1.05 | 1.38 | 1.30 | 1.03 | 0.85 | 1.19 | 0.51 | 0.21 | 0.08 | 0.06 | 0.19 | 0.00 | 0.00 | 0.00 |
| 8 | WT | 1.00 | 1.59 | 1.61 | 1.49 | 1.54 | 1.41 | 0.60 | 0.50 | 0.41 | 0.17 | 0.23 | 0.24 | 0.12 | 0.00 | 0.00 |
| | Mean | 1.0 | 1.3 | 1.2 | 1.2 | 1.2 | 1.1 | 0.9 | 0.7 | 0.6 | 0.6 | 0.4 | 0.3 | 0.2 | 0.1 | 0.1 |
| | Std.dev. | 0.0 | 0.2 | 0.3 | 0.3 | 0.3 | 0.4 | 0.4 | 0.3 | 0.3 | 0.3 | 0.3 | 0.2 | 0.3 | 0.3 | 0.2 |
| 2 | | Measuring day (Wound areas relative to Day 1) | | | | | | | | | | | | | | |
| Animal | Strain | 1 | 2 | 3 | 4 | 5 | 6 | 7 | 8 | 9 | 10 | 11 | 12 | 13 | 14 | 15 |
| 9 | DB | 1.00 | 1.09 | 1.28 | 0.93 | 0.80 | 0.85 | 0.65 | 0.73 | 0.75 | 0.66 | 0.62 | 0.56 | 0.27 | 0.04 | 0.00 |
| 10 | DB | 1.00 | 2.08 | 1.85 | 1.97 | 1.85 | 1.97 | 1.87 | 1.66 | 1.29 | 1.62 | 1.82 | 0.88 | 0.93 | 0.30 | 0.19 |
| 11 | DB | 1.00 | (Dead) | — | — | — | — | — | — | — | — | — | — | — | — | — |
| 12 | DB | 1.00 | 1.09 | 1.40 | 1.50 | 1.51 | 1.35 | 0.69 | 1.01 | 1.04 | 1.06 | 0.99 | 0.81 | 0.44 | 0.22 | 0.18 |
| 13 | DB | 1.00 | 1.30 | 1.33 | 1.50 | 1.74 | 1.51 | 1.48 | 1.46 | 1.45 | 1.45 | 0.94 | 0.30 | 0.31 | 0.00 | 0.00 |
| 14 | DB | 1.00 | 0.97 | 0.91 | 1.03 | 0.84 | 0.78 | 0.93 | 1.01 | 0.69 | 0.69 | 0.82 | 0.36 | 0.00 | 0.00 | 0.00 |
| 15 | DB | 1.00 | 0.76 | 0.93 | 0.99 | 0.97 | 0.91 | 0.95 | 1.28 | 1.25 | 1.08 | 0.57 | 0.78 | 0.54 | 0.15 | 0.14 |
| 16 | DB | 1.00 | 1.13 | 0.80 | 0.70 | 0.66 | 0.46 | 0.53 | 0.55 | 0.33 | 0.21 | — | 0.44 | 0.00 | 0.00 | 0.00 |
| | Mean | 1.0 | 1.2 | 1.2 | 1.2 | 1.2 | 1.1 | 1.0 | 1.1 | 0.9 | 0.9 | 0.9 | 0.6 | 0.2 | 0.0 | 0.0 |
| | Std.dev. | 0.0 | 0.4 | 0.4 | 0.4 | 0.5 | 0.5 | 0.5 | 0.4 | 0.4 | 0.5 | 0.5 | 0.2 | 0.3 | 0.1 | 0.1 |
| 3 | | Measuring day (Wound areas relative to Day 1) | | | | | | | | | | | | | | |
| Animal | Strain | 1 | 2 | 3 | 4 | 5 | 6 | 7 | 8 | 9 | 10 | 11 | 12 | 13 | 14 | 15 |
| 17 | DB | 1.00 | 0.87 | 1.31 | 2.11 | 1.77 | 2.13 | 2.09 | 2.31 | 0.49 | 0.36 | — | 0.59 | 0.00 | 0.00 | 0.00 |
| 18 | DB | 1.00 | 1.81 | 1.09 | 1.25 | 1.08 | 1.15 | 1.04 | 1.33 | 1.57 | 1.32 | 0.37 | 0.24 | 0.00 | 0.00 | 0.00 |
| 19 | DB | 1.00 | (Dead) | — | — | — | — | — | — | — | — | — | — | — | — | — |
| 20 | DB | 1.00 | 2.02 | 2.26 | 2.04 | 2.05 | 1.86 | 1.82 | 1.37 | 1.57 | 1.55 | 1.26 | 1.92 | 0.43 | 0.00 | 0.00 |
| 21 | DB | 1.00 | 1.00 | 1.47 | 1.62 | 1.42 | 1.18 | 1.32 | 1.05 | 1.06 | 0.97 | 0.21 | 0.15 | 0.00 | 0.00 | 0.00 |
| 22 | DB | 1.00 | 0.91 | 1.11 | 1.77 | 1.93 | 1.92 | 1.61 | 1.73 | 1.25 | 1.19 | 0.27 | 0.44 | 0.00 | 0.00 | 0.00 |
| 23 | DB | 1.00 | 0.82 | 0.85 | 1.17 | 1.09 | 0.99 | 0.99 | 0.56 | 0.36 | 0.32 | 0.18 | 0.20 | 0.00 | 0.00 | 0.00 |
| 24 | DB | (Dead) | — | — | — | — | — | — | — | — | — | — | — | — | — | — |
| | Mean | 1.0 | 1.2 | 1.3 | 1.5 | 1.4 | 1.4 | 1.3 | 1.2 | 0.9 | 0.9 | 0.4 | 0.4 | 0.0 | 0.0 | 0.0 |
| | Std.dev. | 0.0 | 0.5 | 0.5 | 0.4 | 0.4 | 0.5 | 0.4 | 0.6 | 0.5 | 0.5 | 0.5 | 0.7 | 0.2 | 0.0 | 0.0 |
| 4 | | Measuring day (Wound areas relative to Day 1) | | | | | | | | | | | | | | |
| Animal | Strain | 1 | 2 | 3 | 4 | 5 | 6 | 7 | 8 | 9 | 10 | 11 | 12 | 13 | 14 | 15 |
| 25 | DB | 1.00 | 0.86 | 0.81 | 0.72 | 0.74 | 0.69 | 0.61 | 0.72 | 0.62 | 0.53 | 0.43 | 0.36 | 0.17 | 0.18 | 0.11 |
| 26 | DB | 1.00 | 0.84 | 1.04 | 1.59 | 1.88 | 1.83 | 1.47 | 1.09 | 0.57 | 0.40 | 0.00 | 0.00 | 0.00 | 0.00 | 0.00 |
| 27 | DB | 1.00 | 1.24 | 1.21 | 1.20 | 1.29 | 1.20 | 1.16 | 1.70 | 0.63 | 0.27 | 0.09 | 0.17 | 0.00 | 0.00 | 0.00 |
| 28 | DB | 1.00 | 0.81 | 1.29 | 1.14 | 1.19 | 1.19 | 1.42 | 1.34 | 0.87 | 0.54 | 0.45 | 0.78 | 0.00 | 0.00 | 0.00 |
| 29 | DB | 1.00 | 0.83 | 0.85 | 0.75 | 0.60 | 0.72 | 0.83 | 0.54 | 0.44 | 0.43 | 0.52 | 0.12 | 0.00 | 0.00 | 0.00 |
| 30 | DB | 1.00 | 1.23 | 1.60 | (Dead) | — | — | — | — | — | — | — | — | — | — | — |
| 31 | DB | 1.00 | 0.71 | 0.86 | 0.86 | 0.80 | 0.89 | 0.87 | 0.91 | 0.31 | 0.18 | 0.14 | 0.30 | 0.00 | 0.00 | 0.00 |
| 32 | DB | (Dead) | — | — | — | — | — | — | — | — | — | — | — | — | — | — |
| | Mean | 1.0 | 0.9 | 1.0 | 1.0 | 1.0 | 1.1 | 1.1 | 1.1 | 0.6 | 0.4 | 0.3 | 0.3 | 0.0 | 0.0 | 0.0 |
| | Std.dev. | 0.0 | 0.2 | 0.3 | 0.3 | 0.5 | 0.4 | 0.3 | 0.4 | 0.2 | 0.1 | 0.2 | 0.3 | 0.1 | 0.1 | 0.0 |
| 5 | | Measuring day (Wound areas relative to Day 1) | | | | | | | | | | | | | | |
| Animal | Strain | 1 | 2 | 3 | 4 | 5 | 6 | 7 | 8 | 9 | 10 | 11 | 12 | 13 | 14 | 15 |
| 33 | DB | 1.00 | 1.22 | 1.27 | 1.44 | 1.34 | 1.27 | 0.95 | 0.99 | 0.97 | 0.49 | 0.42 | 0.76 | 0.00 | 0.00 | 0.00 |
| 34 | DB | 1.00 | 1.08 | 1.49 | 1.70 | 1.57 | 1.60 | 1.48 | 0.38 | 0.31 | 0.40 | 0.35 | 0.29 | 0.14 | 0.00 | 0.00 |
| 35 | DB | 1.00 | 1.50 | 2.34 | 2.24 | 2.45 | 2.47 | 2.47 | 2.24 | 2.18 | 2.66 | 2.27 | 0.43 | 0.15 | 0.00 | 0.00 |
| 36 | DB | 1.00 | 1.03 | 1.52 | 1.49 | 1.50 | 1.42 | 1.46 | 1.42 | 1.67 | 1.56 | 0.33 | 0.14 | 0.00 | 0.00 | 0.00 |
| 37 | DB | 1.00 | 0.96 | 1.41 | 1.13 | 1.04 | 1.28 | 1.51 | 1.51 | 0.84 | 0.83 | 0.82 | 0.81 | 0.83 | 0.81 | 0.60 |
| 38 | DB | 1.00 | 1.17 | 1.76 | 1.47 | 1.37 | 1.48 | 1.44 | 1.41 | 1.64 | 1.60 | 0.94 | 0.64 | 0.00 | 0.00 | 0.00 |
| 39 | DB | 1.00 | (Dead) | — | — | — | — | — | — | — | — | — | — | — | — | — |
| 40 | DB | 1.00 | 0.87 | 1.64 | 1.46 | 1.43 | 1.38 | 1.35 | 1.05 | 0.96 | 0.93 | 1.16 | 0.83 | 0.59 | 0.62 | 0.58 |
| | Mean | 1.0 | 1.1 | 1.6 | 1.5 | 1.5 | 1.5 | 1.4 | 1.2 | 1.1 | 1.1 | 0.8 | 0.5 | 0.1 | 0.1 | 0.1 |
| | Std.dev. | 0.0 | 0.2 | 0.3 | 0.3 | 0.4 | 0.4 | 0.5 | 0.6 | 0.6 | 0.8 | 0.7 | 0.3 | 0.3 | 0.4 | 0.3 |

All wound measured on the line going from head to tail.
All wounds measured from the edge of the wound (between wound surface and normal skin).

TABLE 5
Animal weights

| | | | Measuring day, weight in g | | | |
|---|---|---|---|---|---|---|
| Animal | Strain | Test article | 1 | 4 | 10 | 15 |
| 1 | WT | Vehicle | 22.6 | 22.8 | 22.6 | 23.7 |
| 2 | WT | | 17.6 | 18.5 | 18.6 | 19.4 |
| 3 | WT | | 22.1 | 22.8 | 22.1 | 22.8 |
| 4 | WT | | 20.5 | 21.2 | 21.0 | 21.5 |
| 5 | WT | | 22.3 | 24.5 | 23.6 | 24.1 |
| 6 | WT | | 23.0 | 23.0 | 22.7 | 23.3 |
| 7 | WT | | 21.7 | 22.4 | 21.8 | 22.8 |
| 8 | WT | | 23.5 | 24.8 | 24.5 | 25.7 |
| | | Mean | 21.7 | 22.5 | 22.1 | 22.9 |
| | | Std.dev. | 1.9 | 2.0 | 1.8 | 1.9 |
| 9 | DB | Vehicle | 52.4 | 52.4 | 52.5 | 52.8 |
| 10 | DB | | 46.5 | 46.6 | 44.7 | 44.0 |
| 11 | DB | | 41.5 | (Dead) | — | — |
| 12 | DB | | 53.6 | 51.8 | 50.4 | 49.0 |
| 13 | DB | | 40.1 | 38.7 | 37.8 | 36.9 |
| 14 | DB | | 40.8 | 39.9 | 39.0 | 38.5 |
| 15 | DB | | 43.8 | 43.4 | 40.6 | 38.9 |
| 16 | DB | | 48.9 | 48.4 | 46.0 | 46.7 |
| | | Mean | 46.0 | 45.9 | 44.4 | 43.8 |
| | | Std.dev. | 5.3 | 5.4 | 5.6 | 6.0 |
| 17 | DB | Osteopontin | 53.1 | 52.2 | 50.5 | 50.4 |
| 18 | DB | 0.1% | 46.7 | 47.3 | 47.7 | 47.7 |
| 19 | DB | | 48.8 | (Dead) | — | — |
| 20 | DB | | 48.0 | 48.4 | 46.0 | 47.1 |
| 21 | DB | | 47.9 | 48.6 | 48.2 | 46.2 |
| 22 | DB | | 50.5 | 50.5 | 49.7 | 49.3 |
| 23 | DB | | 42.6 | 42.6 | 40.3 | 39.0 |
| 24 | DB | | (Dead) | — | — | — |
| | | Mean | 48.2 | 48.3 | 47.1 | 46.6 |
| | | Std.dev. | 3.3 | 3.3 | 3.7 | 4.0 |
| 25 | DB | Osteopontin | 41.6 | 41.8 | 41.4 | 40.8 |
| 26 | DB | 1% | 49.4 | 48.8 | 46.8 | 46.5 |
| 27 | DB | | 47.9 | 48.2 | 46.3 | 46.4 |
| 28 | DB | | 45.9 | 45.1 | 44.3 | 44.0 |
| 29 | DB | | 38.8 | 39.2 | 39.1 | 36.2 |
| 30 | DB | | 34.4 | (Dead) | — | — |
| 31 | DB | | 59.3 | 58.9 | 58.7 | 60.3 |
| 32 | DB | | (Dead) | — | — | — |
| | | Mean | 45.3 | 47.0 | 46.1 | 45.7 |
| | | Std.dev. | 8.1 | 6.9 | 6.8 | 8.1 |
| 33 | DB | Osteopontin | 55.9 | 56.6 | 57.1 | 57.2 |
| 34 | DB | 10% | 46.6 | 46.8 | 47.4 | 47.7 |
| 35 | DB | | 47.8 | 48.2 | 48.2 | 47.3 |
| 36 | DB | | 50.9 | 51.9 | 52.7 | 53.9 |
| 37 | DB | | 50.6 | 51.1 | 49.3 | 49.3 |
| 38 | DB | | 47.7 | 47.8 | 45.6 | 44.3 |
| 39 | DB | | 37.7 | (Dead) | — | — |
| 40 | DB | | 45.4 | 44.8 | 40.9 | 37.4 |
| | | Mean | 47.8 | 49.6 | 48.7 | 48.2 |
| | | Std.dev. | 5.2 | 3.9 | 5.2 | 6.4 |

II. Accelerated wound healing in a wound infection model

Introduction

Objective:

The purpose of the study was to test the pharmacological efficacy of Osteopontin on wound healing in a wound infection model in mice.

This part of the report presents data and results for the infected mice only.

Resume:

4 groups of 8 C57BU6J mice were anesthetized, shaved and had an 8mm wound made on their back with a 8 mm punch biopsy instrument at Day 1.

The animals then had their wounds infected with 50pi PBS containing 1x10⁵ CFU of Staphylococcus aureus.

All infected wounds were then covered with “Compeel” blister casts. The casts could be opened for treatment with salves.

Treatment started on Day 1. Groups were treated as follows:

Group 4: +infection; vehicle salve.

Group 5: +infection; 0.5% OPN salve.

Group 6: +infection; 1.0% OPN salve.

Group 7: +infection; 1.5% OPN salve.

Treatment was repeated daily for 15 days. At the same time wounds were scored and measured from Day 1 to Day 15.

Treatment of infected wounds with 1.5% osteopontin in salve had a clear positive effect on wound healing as reflected by reduced wound scores and faster healing wounds. No effects were observed with 0.1 and 1.0% osteopontin in salves.

Justification:

Infected wounds are commonplace at hospitals after surgery, burn wounds and the like. It is especially relevant to have efficient treatment options when dealing with immuno-compromised patients.

Test Article

Description, identification and storage:

| | |
|---|---|
| Test article: | Osteopontin, topical application, 0.5%, 1.0% and 1.5% |
| Vehicle: | Carrier salve |

Preparation:

Pipeline prepared stock and dose solution of Osteopontin in salve.

The stock solution was made at the beginning of the study. The dose solutions were stored in the refrigerator.

Formulation analysis:

Not performed

Concentration and Storage, Stability and Homogeneity:

Osteopontin powder was stored at room temperature.

Stock solution of Osteopontin was stored in refrigerator.

Test system

Species, Strain and Supplier

The study was performed in 32 female C57BU6J mice, 7-8 weeks, from M&B-Taconic.

Environment

The mice were housed 4 in a cage in standard Macrolon cages type 2. Bedding was changed once a week in a laminar flow unit. Temperature was 20° C. to 24° C., and was controlled via the ambient ventilation system in the laboratory. Light cycle was 12-hour dark and 12-hour light (lights on 06.00).

Diet and Water

Diet was Altromin 1314 diet. Water was UV-sterilized and water bottles were refilled when necessary during acclimatization and experiment. Diet and water was administered ad libitum.

Animal Health and Welfare

The animals had FELASA SPF-status and the housing and changing system was designed to assure that the SPF-status was preserved during the study. Educated personnel under veterinary supervision handled the animals. Daily records and decisions were made concerning animal welfare.

Pre-experimental Procedures

Acclimatization and health procedures

Animals were acclimatized for approximately 7 days.

Bacterial culture

Staphylococcus aureus (Pipeline wild type strain). Bacterial concentration: 2×10⁶ cfu/mL

Experimental procedures

Grouping

On Day 1, the mice were grouped and dosed according to the following set-up:

| | | Inoculation load | |
|---|---|---|---|
| Group | Animals | CFU (in 50 μl PBS) | Treatment |
| 4 | 25-32 | 1 × 10⁵ | Vehicle |
| 5 | 33-40 | 1 × 10⁵ | Osteopontin 0.5% |
| 6 | 41-48 | 1 × 10⁵ | Osteopontin 1.0% |
| 7 | 49-56 | 1 × 10⁵ | Osteopontin 1.5% |

Bacterial concentration: 2×10⁶ cfu/mL

Analgesia

All mice were subjected to analgesia by administration of 5 mg/kg s.c. Rimadryl 1 hour before wound procedures.

Introduction of wounds

One hour after dosing of Rimadryl and approximately 15 minutes after surgical anesthesia was induced, one wound was induced on each mouse.

One wound was introduced on the dorsal skin of each mouse by the following procedure:

1. The back of the mouse was shaven in a 2×3 cm area.

2. The 8 ml punch biopsy instrument was used to make a circular cut in the center area of the shaven area.

3. The skin piece was lifted a bit and carefully dissected free from the mouse.

This was followed by inoculation of the wounds by Staph. aureus according to the following procedure:

1. 50 pl of PBS with the bacterial culture was placed within the wound area by a pipette.

2. All wounds were covered with “Compeel” blister casts.

3. The mice were then-ready for treatment.

Treatment

Treatment started on Day 1.

A window was cut in the “Compeel” casts of the mice.

The salves were then applied to all treatment groups as described in section 5.1.

Group 4 was treated with carrier salve, Group 5 was treated with 0.5% OPN in salve, Group 6 was treated with 1.0% OPN in salve and Group 7 was treated with 1.5% OPN in salve. The salves were carefully applied topically to the wounds.

Treatment was repeated daily for 15 days.

Observations and measurements

The wounds were scored and measured from Days 1-15.

The diameters of the wounds were measured longitudinally (on the line running from head to tail) and recorded each day.

The appearance of the wounds were scored and recorded each day according to the following system:

0: Completely healed

1: Small wound still detected.

2: No scab but still wound

3: No original scab but new scab (old scab fallen off but new scab created)

4: original scab still on wound.

Body weights were recorded on Days 1, 5, 8, 12 and 15.

Termination

At termination the mice were euthanized.

Study overview

| Day | Date | |
|---|---|---|
| −7 | 03.03.2005 | Animals arrive at Pipeline Biotech |
| | | Acclimatization |
| 1 | 10.03.2005 | Introduction of wounds. |
| | | Inoculation of wounds with Staph. aureus. |
| | | Cover wounds by “Compeel” casts |
| | | Treatment, measurements and scoring |
| 2 | | Treatment, measurements and scoring |
| 3 | | Treatment, measurements and scoring |
| 4 | | Treatment, measurements and scoring, body weights |
| 5 | | Treatment, measurements and scoring |
| 6 | | Treatment, measurements and scoring |
| 7 | | Treatment, measurements and scoring, body weights |
| 8 | | Treatment, measurements and scoring |
| 9 | | Treatment, measurements and scoring |
| 10 | | Treatment, measurements and scoring |
| 11 | | Treatment, measurements and scoring, body weights |
| 12 | | Treatment, measurements and scoring |
| 13 | | Treatment, measurements and scoring |
| 14 | | Treatment, measurements and scoring |
| 15 | 24.03.2004 | Treatment, measurements and scoring, body weights |
| | | Termination |

Results

Wound Scores

Results are shown in Table 1 (appendix).

Data were analysed statistically by the Mann-Whitney paired Rank-sum test (Wilcoxon two sample test). Results in test groups were compared to Group 4. Results of the statistical analysis were as follows:

| | | | | | Day | Day | Day | Day | Day | Day |
|---|---|---|---|---|---|---|---|---|---|---|
| Group | Day 6 | Day 7 | Day 8 | Day 9 | 10 | 11 | 12 | 13 | 14 | 15 |
| 4 | — | — | — | — | — | — | — | — | — | — |
| 5 | ns | ns | ns | ns | ns | ns | ns | ns | ns | ns |
| 6 | ns | ns | ns | ns | ns | ns | ns | 0.05 | ns | ns |
| 7 | ns | 0.05 | ns | ns | ns | 0.02 | 0.05 | 0.01 | ns | 0.05 |

n.s.: non significant

Conclusions:

| | | |
|---|---|---|
| Group 4: | +infection; vehicle salve. | |
| Group 5: | +infection; 0.5% OPN salve. | Not different from Gr. 4 |
| Group 6: | +infection; 1.0% OPN salve. | Significantly lower scores |
| | | than Gr. 4 on day 13 |
| Group 7: | +infection; 1.5% OPN salve. | Significantly lower scores |
| | | than Gr. 4 on days 7, 11, |
| | | 12, 13 and 15 |

Wound Diameters

Results are shown in Table 2 (appendix).

Data were analysed statistically by paired F- and Student t-tests. Results in test groups were compared to Group 4. Results of the statistical analysis were as follows:

| | | | | | | | | | | Day | | | | | |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | Day 11 | Day 12 | Day 13 | Day 14 | Day 15 |
| 4 | — | — | — | — | — | — | — | — | — | — | — | — | — | — | — |
| 5 | ns | <0.01 | <0.05 | <0.05 | ns | ns | ns | ns | ns | ns | ns | ns | <0.05 | ns | ns |
| 6 | ns | <0.05 | <0.05 | ns | ns | ns | ns | ns | ns | ns | ns | ns | ns | ns | ns |
| 7 | ns | ns | ns | ns | ns | ns | ns | <0.01 | ns | ns | <0.05 | <0.01 | <0.01 | ns | <0.001 |

Results of AUC (Area Under Curve) statistical analysis of Wound diameters are as follows:
(Results in test groups were compared to Group 4.)
Group 5: Non significant.
Group 6: Non significant.
Group 7: Non significant.

Conclusions:

| | | |
|---|---|---|
| Group 4: | +infection; vehicle salve. | |
| Group 5: | +infection; 0.5% OPN | Sign. smaller diameters on Day |
| | salve. | 13 than Group 4. But generally |
| | | not sign. smaller diameters than |
| | | group 4. |
| Group 6: | +infection; 1.0% OPN | Generally not sign. smaller |
| | salve. | diameters than Group 4. |
| Group 7: | +infection; 1.5% OPN | Sign. smaller diameters on Days |
| | salve. | 8, 11, 12 13 and 15 than |
| | | Group 4. But generally not sign. |
| | | smaller diameters than group 4. |

Wound Areas

Results are shown in Table 3 (appendix).

Data were analysed statistically by paired F- and Student t-tests. Results in test groups were compared to Group 4. Results of the statistical analysis were as follows:

| | | | | | | | | | | Day | | | | | |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | Day 11 | Day 12 | Day 13 | Day 14 | Day 15 |
| 4 | — | — | — | — | — | — | — | — | — | — | — | — | — | — | — |
| 5 | ns | <0.01 | <0.05 | <0.05 | ns | ns | ns | ns | ns | ns | ns | ns | ns | ns | ns |
| 6 | ns | <0.05 | <0.05 | ns | ns | ns | ns | ns | ns | ns | ns | ns | ns | ns | ns |
| 7 | ns | ns | ns | ns | ns | ns | ns | <0.01 | ns | ns | <0.05 | <0.05 | <0.05 | ns | ns |

Results of AUC (Area Under Curve) statistical analysis of Wound Areas are as follows:
(Results in test groups were compared to Group 4.)
Group 5: Non significant.
Group 6: Non significant.
Group 7: Non significant.

Conclusions:

| | | |
|---|---|---|
| Group 4: | +infection; vehicle salve. | |
| Group 5: | +infection; 0.5% OPN | Generally not sign. smaller areas |
| | salve. | than Group 4. |
| Group 6: | +infection; 1.0% OPN | Generally not sign. smaller areas |
| | salve. | than Group 4. |
| Group 7: | +infection; 1.5% OPN | Sign. smaller diameters on Days |
| | salve. | 8, 11, 12 and 13 than Group 4. |
| | | But generally not sign. smaller |
| | | areas than group 4. |

Wound Areas relative to Day 1

Results are shown in Table 4 (appendix).

Data were analysed statistically by paired F- and Student t-tests. Results in test groups were compared to Group 4. Results of the statistical analysis were as follows:

| | | | | | | | | | | Day | Day | Day | Day | Day | Day |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | 11 | 12 | 13 | 14 | 15 |
| 4 | — | — | — | — | — | — | — | — | — | — | — | — | — | — | — |
| 5 | — | <0.05 | ns | ns | ns | ns | ns | ns | ns | ns | ns | ns | ns | ns | ns |
| 6 | — | ns | ns | ns | ns | ns | ns | ns | ns | ns | ns | ns | ns | ns | ns |
| 7 | — | ns | ns | ns | ns | ns | ns | <0.05 | ns | ns | ns | <0.05 | ns | ns | ns |

Results of AUC (Area Under Curve) statistical analysis of Relative Wound Areas are as follows:
(Results in test groups were compared to Group 4.)
Group 5: Non significant.
Group 6: Non significant.
Group 7: <0.05

Conclusions:

| | | |
|---|---|---|
| Group 4: | +infection; vehicle salve. | |
| Group 5: | +infection; 0.5% OPN salve. | Generally not sign. smaller |
| | | areas than Group 4. |
| Group 6: | +infection; 1.0% OPN salve. | Generally not sign. smaller |
| | | areas than Group 4. |
| Group 7: | +infection; 1.5% OPN salve. | Generally sign. smaller areas |
| | | than Group 4. |

Conclusion

Treatment with 1.5% osteopontin salve clearly decreased wound scores of infected wounds during the last week of treatment. Thus 1.5% osteopontin salve had a positive effect on healing of these wounds. The same positive effect was only observed on Day 13 for animals treated with 1.0% osteopontin salve. No effect was observed with 0.1% Osteopontin salve. 1.5% osteopontin salve also had a marked positive effect on wound size, as reflected in wound diameters, wound areas and relative wound areas. The same positive effect was not observed with lower concentrations of osteopontin in salves.

Archive

The final report as well as all raw data and results are kept in the archives of Pipeline Biotech A/S for a period of five (5) years from the end of the study.

TABLE 1
Wound scores:

| 4 | Scoring day | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 25 | 4 | 4 | 4 | 4 | 4 | 2 | 2.5 | 2.5 | 3 | 3 | 3 | 2 | 2 | 0 | 0 |
| 26 | 4 | 4 | 4 | 4 | 4 | 4 | 4 | 4 | 3 | 3 | 3 | 3 | 3 | 0 | 1 |
| 27 | 4 | 4 | 4 | 4 | 4 | 4 | 4 | 4 | 4 | 4 | 3 | 3 | 3 | 3 | 3 |
| 28 | 4 | 4 | 4 | 4 | 4 | 4 | 4 | 3 | 3 | 3 | 3 | 2 | 2 | 1 | 1 |
| 29 | 4 | 4 | 4 | 4 | 4 | 4 | 4 | 3 | 3 | 3 | 3 | 2 | 2 | 1 | 2 |
| 30 | 4 | 4 | 4 | 4 | 4 | 4 | 4 | 4 | 4 | 4 | 3 | 3 | 3 | 3 | 3 |
| 31 | 4 | 4 | 4 | 4 | 4 | 4 | 4 | 4 | 3 | 3 | 3 | 2 | 2 | 0 | 0 |
| 32 | 4 | 4 | 4 | 4 | 4 | 4 | 2.5 | 3 | 3 | 3 | 2 | 1 | 1 | 0 | 1 |
| Mean | 4.0 | 4.0 | 4.0 | 4.0 | 4.0 | 3.8 | 3.6 | 3.4 | 3.3 | 3.3 | 2.9 | 2.3 | 2.3 | 1.0 | 1.4 |
| Std.dev | — | — | — | — | — | 0.7 | 0.7 | 0.6 | 0.5 | 0.5 | 0.4 | 0.7 | 0.7 | 1.3 | 1.2 |
| 5 | Scoring day | | | | | | | | | | | | | | |
| Animal | 1 | 2 | 3 | 4 | 5 | 6 | 7 | 8 | 9 | 10 | 11 | 12 | 13 | 14 | 15 |
| 33 | 4 | 4 | 4 | 4 | 4 | 4 | 4 | 4 | 3 | 3 | 3 | 3 | 3 | 3 | 1 |
| 34 | 4 | 4 | 4 | 4 | 4 | 4 | 3 | 3 | 3 | 3 | 3 | 2 | 2 | 0 | 0 |
| 35 | 4 | 4 | 4 | 4 | 4 | 4 | 4 | 4 | 3 | 3 | 3 | 3 | 3 | 0 | 0 |
| 36 | 4 | 4 | 4 | 4 | 4 | 4 | 4 | 3 | 3 | 3 | 3 | 3 | 1 | 0 | 0 |
| 37 | 4 | 4 | 4 | 4 | 4 | 4 | 3 | 3 | 3 | 3 | 3 | 3 | 2 | 1 | 1 |
| 38 | 4 | 4 | 4 | 4 | 4 | 4 | 3 | 3 | 3 | 3 | 3 | 3 | 2 | 1 | 0 |
| 39 | 4 | 4 | 4 | 4 | 4 | 4 | 4 | 4 | 4 | 3.5 | 3 | 3 | 3 | 2 | 1 |
| 40 | 4 | 4 | 4 | 4 | 4 | 4 | 4 | 3 | 3 | 3 | 3 | 3 | 1.5 | 0 | 0 |
| Mean | 4.0 | 4.0 | 4.0 | 4.0 | 4.0 | 4.0 | 3.6 | 3.4 | 3.1 | 3.1 | 3.0 | 2.9 | 2.2 | 0.9 | 0.4 |
| Std.dev | — | — | — | — | — | — | 0.5 | 0.5 | 0.4 | 0.2 | — | 0.4 | 0.8 | 1.1 | 0.5 |
| 6 | Scoring day | | | | | | | | | | | | | | |
| Animal | 1 | 2 | 3 | 4 | 5 | 6 | 7 | 8 | 9 | 10 | 11 | 12 | 13 | 14 | 15 |
| 41 | 4 | 4 | 4 | 4 | 4 | 2.5 | 2.5 | 3 | 0 | 0 | 0 | 0 | 0 | 0 | 0 |
| 42 | 4 | 4 | 4 | 4 | 4 | 4 | 2.5 | 2.5 | 2 | 2 | 2 | 1 | 0 | 0 | 0 |
| 43 | 4 | 4 | 4 | 4 | 4 | 2 | 2.5 | 3 | 3 | 3 | 2 | 1 | 0 | 0 | 0 |
| 44 | 4 | 4 | 4 | 4 | 4 | 4 | 4 | 4 | 4 | 4 | 3 | 3 | 2 | 2 | 1 |
| 45 | 4 | 4 | 4 | 4 | 4 | 4 | 4 | 3 | 3 | 3 | 3 | 3 | 2 | 0 | 0 |
| 46 | 4 | 4 | 4 | 4 | 4 | 4 | 4 | 4 | 4 | 4 | 4 | 4 | 3 | 3 | 3 |
| 47 | 4 | 4 | 4 | 4 | 4 | 2 | 2 | 3 | 3 | 3 | 3 | 1 | 0 | 0 | 0 |
| 48 | 4 | 4 | 4 | 4 | 4 | 4 | 2.5 | 3 | 3 | 2 | 2 | 0 | 0 | 0 | 0 |
| Mean | 4.0 | 4.0 | 4.0 | 4.0 | 4.0 | 3.3 | 3.0 | 3.2 | 2.8 | 2.6 | 2.4 | 1.6 | 0.9 | 0.6 | 0.5 |
| Std.dev | — | — | — | — | — | 1.0 | 0.8 | 0.5 | 1.3 | 1.3 | 1.2 | 1.5 | 1.2 | 1.2 | 1.1 |

Scoring:
0: Completely healed
1: Small wound still detected.
2: No scab but still wound
3: No original scab but new scab (old scab fallen off but new scab created)
4: original scab still on wound.

TABLE 2
Wound Diameters

| 4 | Measuring day (mm) | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 25 | 8.18 | 4.29 | 4.20 | 4.35 | 4.49 | 4.65 | 4.54 | 4.28 | 2.89 | 2.75 | 2.47 | 1.23 | 1.19 | 0 | 0 |
| 26 | 3.96 | 4.07 | 3.73 | 4.06 | 3.86 | 6.21 | 6.18 | 6.29 | 3.48 | 2.54 | 3.00 | 2.03 | 2.10 | 0 | 2.31 |
| 27 | 6.34 | 5.52 | 5.43 | 4.59 | 3.41 | 5.50 | 6.01 | 5.99 | 6.20 | 5.35 | 5.83 | 4.49 | 4.50 | 3.00 | 3.23 |
| 28 | 10.42 | 8.86 | 8.36 | 10.53 | 10.71 | 10.84 | 10.16 | 8.59 | 5.62 | 4.96 | 4.99 | 3.04 | 3.35 | 0.85 | 0.97 |
| 29 | 7.00 | 5.57 | 5.36 | 7.41 | 8.80 | 7.26 | 7.03 | 6.68 | 5.19 | 3.45 | 3.27 | 2.42 | 1.98 | 2.00 | 2.19 |
| 30 | 5.84 | 5.34 | 5.13 | 5.92 | 5.55 | 6.61 | 7.01 | 7.10 | 7.19 | 10.42 | 5.91 | 4.96 | 5.30 | 5.05 | 4.63 |
| 31 | 8.00 | 8.09 | 8.24 | 8.10 | 9.06 | 8.12 | 7.70 | 7.83 | 3.90 | 3.60 | 3.53 | 3.29 | 1.00 | 0 | 0 |
| 32 | 6.25 | 4.49 | 3.77 | 4.75 | 5.00 | 5.44 | 3.12 | 3.30 | 0.96 | 1.73 | 1.71 | 1.23 | 0.82 | 0 | 1.52 |
| Mean | 7.0 | 5.8 | 5.5 | 6.2 | 6.4 | 6.8 | 6.5 | 6.3 | 4.4 | 4.4 | 3.8 | 2.8 | 2.5 | 1.4 | 1.9 |
| Std.dev | 1.9 | 1.8 | 1.8 | 2.3 | 2.8 | 2.0 | 2.1 | 1.8 | 2.0 | 2.7 | 1.6 | 1.4 | 1.7 | 1.9 | 1.6 |
| 5 | Measuring day (mm) | | | | | | | | | | | | | | |
| Animal | 1 | 2 | 3 | 4 | 5 | 6 | 7 | 8 | 9 | 10 | 11 | 12 | 13 | 14 | 15 |
| 33 | 10.03 | 9.67 | 8.71 | 9.72 | 9.40 | 8.59 | 9.27 | 9.66 | 3.33 | 4.02 | 4.41 | 2.98 | 3.78 | 3.654 | 1.65 |
| 34 | 10.19 | 9.54 | 10.28 | 10.93 | 11.13 | 9.50 | 6.34 | 5.39 | 4.00 | 4.17 | 4.16 | 1.99 | 0.84 | 0 | 0 |
| 35 | 6.24 | 7.15 | 7.25 | 7.00 | 8.06 | 7.95 | 7.88 | 7.50 | 4.04 | 3.49 | 3.68 | 3.35 | 1.24 | 0 | 0 |
| 36 | 8.81 | 6.70 | 6.25 | 9.02 | 6.70 | 7.71 | 7.40 | 3.85 | 4.04 | 3.48 | 3.38 | 1.62 | 0 | 0 | 0 |
| 37 | 9.25 | 8.19 | 7.54 | 7.33 | 7.72 | 7.10 | 4.30 | 4.51 | 4.59 | 3.56 | 2.89 | 1.59 | 1.02 | 0.98 | 2.01 |
| 38 | 7.24 | 7.82 | 6.31 | 7.07 | 7.11 | 6.32 | 4.51 | 5.01 | 4.50 | 4.52 | 4.02 | 1.73 | 1.72 | 1.51 | 0 |
| 39 | 9.89 | 9.38 | 8.54 | 7.06 | 7.10 | 8.55 | 8.32 | 8.29 | 7.09 | 7.97 | 4.65 | 3.19 | 1.26 | 1.15 | 1.18 |
| 40 | 6.27 | 7.14 | 6.30 | 6.60 | 7.13 | 6.44 | 6.35 | 4.14 | 3.22 | 2.64 | 2.36 | 0.87 | 0 | 0 | 0 |
| Mean | 8.5 | 8.2 | 7.6 | 8.1 | 8.0 | 7.8 | 6.8 | 6.0 | 4.4 | 4.2 | 3.7 | 2.2 | 1.2 | 0.9 | 0.6 |
| Std.dev | 1.7 | 1.2 | 1.4 | 1.6 | 1.5 | 1.1 | 1.8 | 2.2 | 1.2 | 1.6 | 0.8 | 0.9 | 1.2 | 1.3 | 0.9 |
| 6 | Measuring day (mm) | | | | | | | | | | | | | | |
| Animal | 1 | 2 | 3 | 4 | 5 | 6 | 7 | 8 | 9 | 10 | 11 | 12 | 13 | 14 | 15 |
| 41 | 8.63 | 6.47 | 6.62 | 6.99 | 6.69 | 3.68 | 3.21 | 2.86 | 0 | 0 | 0 | 0 | 0 | 0 | 0 |
| 42 | 8.24 | 7.31 | 6.91 | 6.97 | 6.89 | 6.22 | 4.40 | 3.24 | 2.51 | 2.52 | 2.63 | 0.96 | 0 | 0 | 0 |
| 43 | 9.14 | 7.53 | 7.12 | 7.33 | 7.58 | 5.22 | 3.16 | 2.91 | 2.66 | 2.66 | 2.15 | 0.70 | 0 | 0 | 0 |
| 44 | 6.33 | 5.39 | 5.24 | 7.33 | 9.79 | 6.01 | 7.01 | 7.40 | 7.40 | 7.61 | 5.55 | 4.18 | 2.86 | 2.91 | 2.01 |
| 45 | 10.17 | 10.01 | 10.25 | 9.81 | 10.13 | 10.14 | 9.80 | 6.90 | 6.90 | 6.79 | 3.89 | 2.19 | 1.36 | 0 | 0 |
| 46 | 9.50 | 10.51 | 9.16 | 8.80 | 9.22 | 9.57 | 10.74 | 10.89 | 11.19 | 11.91 | 11.30 | 11.45 | 6.47 | 6.77 | 6.96 |
| 47 | 7.00 | 9.08 | 8.84 | 7.09 | 8.68 | 6.15 | 5.91 | 4.17 | 4.17 | 4.31 | 3.05 | 0.68 | 0 | 0 | 0 |
| 48 | 6.57 | 7.40 | 6.96 | 6.93 | 7.01 | 6.90 | 3.78 | 3.78 | 3.78 | 2.67 | 1.90 | 0 | 0 | 0 | 0 |
| Mean | 8.2 | 8.0 | 7.6 | 7.7 | 8.2 | 6.7 | 6.0 | 5.3 | 4.8 | 4.8 | 3.8 | 2.5 | 1.3 | 1.2 | 1.1 |
| Std.dev | 1.4 | 1.8 | 1.6 | 1.1 | 1.4 | 2.2 | 3.0 | 2.9 | 3.5 | 3.8 | 3.4 | 3.9 | 2.3 | 2.5 | 2.5 |
| 7 | Measuring day (mm) | | | | | | | | | | | | | | |
| Animal | 1 | 2 | 3 | 4 | 5 | 6 | 7 | 8 | 9 | 10 | 11 | 12 | 13 | 14 | 15 |
| 49 | 5.47 | 6.15 | 4.74 | 5.02 | 5.06 | 5.24 | 5.14 | 4.03 | 2.93 | 2.60 | 2.53 | 0 | 0 | 0 | 0 |
| 50 | 9.48 | 7.53 | 5.74 | 6.68 | 6.11 | 6.78 | 6.88 | 6.71 | 7.02 | 5.56 | 3.56 | 1.83 | 1.62 | 1.70 | 0 |
| 51 | 6.41 | 6.59 | 6.03 | 8.03 | 6.02 | 7.41 | 8.01 | 5.01 | 4.98 | 3.95 | 3.94 | 1.21 | 0 | 0 | 0 |
| 52 | 7.08 | 6.51 | 5.60 | 6.48 | 6.68 | 6.46 | 2.82 | 2.76 | 1.99 | 1.51 | 1.17 | 0 | 0 | 0 | 0 |
| 53 | 8.05 | 7.09 | 7.97 | 7.15 | 7.60 | 7.51 | 2.98 | 2.96 | 2.99 | 2.16 | 0.96 | 0 | 0 | 0 | 0 |
| 54 | 8.20 | 5.74 | 5.00 | 6.11 | 5.69 | 5.73 | 2.80 | 3.01 | 1.59 | 2.75 | 1.57 | 0.59 | 0 | 0 | 0 |
| 55 | 6.94 | 5.96 | 5.16 | 6.09 | 5.15 | 5.25 | 4.03 | 2.00 | 1.09 | 0.99 | 0.92 | 0 | 0 | 0 | 0 |
| 56 | 9.10 | 7.73 | 7.91 | 8.83 | 7.78 | 7.07 | 5.56 | 5.60 | 4.66 | 4.17 | 3.40 | 2.23 | 0 | 0 | 0 |
| Mean | 7.6 | 6.7 | 6.0 | 6.8 | 6.3 | 6.4 | 4.8 | 4.0 | 3.4 | 3.0 | 2.3 | 0.7 | 0.2 | 0.2 | 0.0 |
| Std.dev | 1.4 | 0.7 | 1.3 | 1.2 | 1.0 | 0.9 | 2.0 | 1.6 | 2.0 | 1.5 | 1.3 | 0.9 | 0.6 | 0.6 | 0.0 |

Wound scores

| 7 | Scoring day | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 49 | 4 | 4 | 4 | 4 | 4 | 4 | 4 | 3 | 3 | 3 | 2 | 2 | 0 | 0 | 0 |
| 50 | 4 | 4 | 4 | 4 | 4 | 4 | 4 | 4 | 4 | 4 | 3 | 3 | 2 | 1 | 1 |
| 51 | 4 | 4 | 4 | 4 | 4 | 4 | 2 | 3 | 3 | 3 | 3 | 1.5 | 0 | 0 | 0 |
| 52 | 4 | 4 | 4 | 4 | 4 | 4 | 2.5 | 3 | 3 | 2 | 1 | 0 | 0 | 0 | 0 |
| 53 | 4 | 4 | 4 | 4 | 4 | 4 | 2 | 2 | 3 | 3 | 1 | 0 | 0 | 0 | 0 |
| 54 | 4 | 4 | 4 | 4 | 4 | 4 | 2 | 2 | 3 | 3 | 2 | 1 | 0 | 0 | 0 |
| 55 | 4 | 4 | 4 | 4 | 4 | 4 | 2 | 2 | 2 | 1 | 1 | 0 | 0 | 0 | 0 |
| 56 | 4 | 4 | 4 | 4 | 4 | 2 | 2 | 3 | 3 | 3 | 1 | 1.5 | 0 | 0 | 0 |
| Mean | 4.0 | 4.0 | 4.0 | 4.0 | 4.0 | 3.8 | 2.6 | 2.8 | 13.0 | 2.8 | 1.8 | 1.1 | 0.3 | 0.1 | 0.1 |
| Std.dev | — | — | — | — | — | 0.7 | 0.9 | 0.7 | 0.5 | 0.9 | 0.9 | 1.1 | 0.7 | 0.4 | 0.4 |

Scoring:
0: Completely healed
1: Small wound still detected.
2: No scab but still wound
3: No original scab but new scab (old scab fallen off but new scab created)
4: original scab still on wound.
All wound measured on the line going from head to tail.
All wounds measured from the edge of the wound (between wound surface and normal skin).

TABLE 3
Wound Areas

| 4 | Measuring day (mm²) | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 25 | 52.53 | 14.45 | 13.85 | 14.85 | 15.83 | 16.97 | 16.18 | 14.38 | 6.56 | 5.94 | 4.79 | 1.19 | 1.11 | 0.00 | 0.00 |
| 26 | 12.31 | 13.00 | 10.92 | 12.94 | 11.70 | 30.27 | 29.98 | 31.06 | 9.51 | 5.06 | 7.07 | 3.23 | 3.46 | 0.00 | 4.19 |
| 27 | 31.55 | 23.92 | 23.15 | 16.54 | 9.13 | 23.75 | 28.35 | 28.17 | 30.18 | 22.47 | 26.68 | 15.83 | 15.90 | 7.07 | 8.19 |
| 28 | 85.23 | 61.62 | 54.86 | 87.04 | 90.04 | 92.24 | 81.03 | 57.92 | 24.79 | 19.31 | 19.55 | 7.25 | 8.81 | 0.57 | 0.74 |
| 29 | 38.47 | 24.35 | 22.55 | 43.10 | 60.79 | 41.38 | 38.80 | 35.03 | 21.14 | 9.34 | 8.39 | 4.60 | 3.08 | 3.14 | 3.76 |
| 30 | 26.77 | 22.38 | 20.66 | 27.51 | 24.18 | 34.30 | 38.57 | 39.57 | 40.58 | 85.23 | 27.42 | 19.31 | 22.05 | 20.02 | 16.83 |
| 31 | 50.24 | 51.38 | 53.30 | 51.50 | 64.44 | 51.76 | 46.54 | 48.13 | 11.94 | 10.17 | 9.78 | 8.50 | 0.79 | 0.00 | 0.00 |
| 32 | 30.66 | 15.83 | 11.16 | 17.71 | 19.63 | 23.23 | 7.64 | 8.55 | 0.72 | 2.35 | 2.30 | 1.19 | 0.53 | 0.00 | 1.81 |
| Mean | 38.5 | 26.2 | 24.0 | 30.3 | 31.8 | 36.6 | 32.8 | 30.7 | 15.4 | 14.9 | 11.6 | 6.3 | 5.0 | 1.5 | 2.7 |
| Std.dev | 22.0 | 18.1 | 17.8 | 25.7 | 30.4 | 24.1 | 22.2 | 16.3 | 13.4 | 27.3 | 9.9 | 6.7 | 8.0 | 7.0 | 5.7 |
| 5 | Measuring day (mm²) | | | | | | | | | | | | | | |
| Animal | 1 | 2 | 3 | 4 | 5 | 6 | 7 | 8 | 9 | 10 | 11 | 12 | 13 | 14 | 15 |
| 33 | 78.97 | 73.40 | 59.55 | 74.17 | 69.36 | 57.92 | 67.46 | 73.25 | 8.70 | 12.69 | 15.27 | 6.97 | 11.22 | 10.48 | 2.14 |
| 34 | 81.51 | 71.44 | 82.96 | 93.78 | 97.24 | 70.85 | 31.55 | 22.81 | 12.56 | 13.65 | 13.58 | 3.11 | 0.55 | 0.00 | 0.00 |
| 35 | 30.57 | 40.13 | 41.26 | 38.47 | 51.00 | 49.61 | 48.74 | 44.16 | 12.81 | 9.56 | 10.63 | 8.81 | 1.21 | 0.00 | 0.00 |
| 36 | 60.93 | 35.24 | 30.66 | 63.87 | 35.24 | 46.66 | 42.99 | 11.64 | 12.81 | 9.51 | 8.97 | 2.06 | 0.00 | 0.00 | 0.00 |
| 37 | 67.17 | 52.65 | 44.63 | 42.18 | 46.78 | 39.57 | 14.51 | 15.97 | 16.54 | 9.95 | 6.56 | 1.98 | 0.82 | 0.75 | 3.17 |
| 38 | 41.15 | 48.00 | 31.26 | 39.24 | 39.68 | 31.35 | 15.97 | 19.70 | 15.90 | 16.04 | 12.69 | 2.35 | 2.32 | 1.79 | 0.00 |
| 39 | 76.78 | 69.07 | 57.25 | 39.13 | 39.57 | 57.39 | 54.34 | 53.95 | 39.46 | 49.86 | 16.97 | 7.99 | 1.25 | 1.04 | 1.09 |
| 40 | 30.86 | 40.02 | 31.16 | 34.19 | 39.91 | 32.56 | 31.65 | 13.45 | 8.14 | 5.47 | 4.37 | 0.59 | 0.00 | 0.00 | 0.00 |
| Mean | 56.6 | 52.8 | 45.9 | 51.4 | 50.8 | 47.4 | 36.3 | 28.7 | 14.9 | 14.1 | 10.7 | 3.7 | 1.2 | 0.7 | 0.3 |
| Std.dev | 21.4 | 15.5 | 18.4 | 21.7 | 21.1 | 13.6 | 18.5 | 22.6 | 10.0 | 14.1 | 4.3 | 3.2 | 3.7 | 3.6 | 1.2 |
| 6 | Measuring day (mm²) | | | | | | | | | | | | | | |
| Animal | 1 | 2 | 3 | 4 | 5 | 6 | 7 | 8 | 9 | 10 | 11 | 12 | 13 | 14 | 15 |
| 41 | 58.46 | 32.86 | 34.40 | 38.36 | 35.13 | 10.63 | 8.09 | 6.42 | 0.00 | 0.00 | 0.00 | 0.00 | 0.00 | 0.00 | 0.00 |
| 42 | 53.30 | 41.95 | 37.48 | 38.14 | 37.27 | 30.37 | 15.20 | 8.24 | 4.95 | 4.99 | 5.43 | 0.72 | 0.00 | 0.00 | 0.00 |
| 43 | 65.58 | 44.51 | 39.80 | 42.18 | 45.10 | 21.39 | 7.84 | 6.65 | 5.55 | 5.55 | 3.63 | 0.38 | 0.00 | 0.00 | 0.00 |
| 44 | 31.45 | 22.81 | 21.55 | 42.18 | 75.24 | 28.35 | 38.57 | 42.99 | 42.99 | 45.46 | 24.18 | 13.72 | 6.42 | 6.65 | 3.17 |
| 45 | 81.19 | 78.66 | 82.47 | 75.55 | 80.55 | 80.71 | 75.39 | 37.37 | 37.37 | 36.19 | 11.88 | 3.76 | 1.45 | 0.00 | 0.00 |
| 46 | 70.85 | 86.71 | 65.87 | 60.79 | 66.73 | 71.89 | 90.55 | 93.09 | 98.29 | 111.35 | 100.24 | 102.92 | 32.86 | 35.98 | 38.03 |
| 47 | 38.47 | 64.72 | 61.34 | 39.46 | 59.14 | 29.69 | 27.42 | 13.65 | 13.65 | 14.58 | 7.30 | 0.36 | 0.00 | 0.00 | 0.00 |
| 48 | 33.88 | 42.99 | 38.03 | 37.70 | 38.57 | 37.37 | 11.22 | 11.22 | 11.22 | 5.60 | 2.83 | 0.00 | 0.00 | 0.00 | 0.00 |
| Mean | 52.8 | 49.8 | 45.8 | 46.0 | 53.4 | 35.6 | 28.3 | 21.8 | 18.3 | 18.2 | 11.4 | 5.0 | 1.4 | 1.1 | 1.0 |
| Std.dev | 18.3 | 22.5 | 20.2 | 13.9 | 18.1 | 24.5 | 32.1 | 30.1 | 32.8 | 37.5 | 33.5 | 35.7 | 11.4 | 12.6 | 13.3 |
| 7 | Measuring day (mm²) | | | | | | | | | | | | | | |
| Animal | 1 | 2 | 3 | 4 | 5 | 6 | 7 | 8 | 9 | 10 | 11 | 12 | 13 | 14 | 15 |
| 49 | 23.49 | 29.69 | 17.64 | 19.78 | 20.10 | 21.55 | 20.74 | 12.75 | 6.74 | 5.31 | 5.02 | 0.00 | 0.00 | 0.00 | 0.00 |
| 50 | 70.55 | 44.51 | 25.86 | 35.03 | 29.31 | 36.09 | 37.16 | 35.34 | 38.69 | 24.27 | 9.95 | 2.63 | 2.06 | 2.27 | 0.00 |
| 51 | 32.25 | 34.09 | 28.54 | 50.62 | 28.45 | 43.10 | 50.37 | 19.70 | 19.47 | 12.25 | 12.19 | 1.15 | 0.00 | 0.00 | 0.00 |
| 52 | 39.35 | 33.27 | 24.62 | 32.96 | 35.03 | 32.76 | 6.24 | 5.98 | 3.11 | 1.79 | 1.07 | 0.00 | 0.00 | 0.00 | 0.00 |
| 53 | 50.87 | 39.46 | 49.86 | 40.13 | 45.34 | 44.27 | 6.97 | 6.88 | 7.02 | 3.66 | 0.72 | 0.00 | 0.00 | 0.00 | 0.00 |
| 54 | 52.78 | 25.86 | 19.63 | 29.31 | 25.42 | 25.77 | 6.15 | 7.11 | 1.98 | 5.94 | 1.93 | 0.27 | 0.00 | 0.00 | 0.00 |
| 55 | 37.81 | 27.88 | 20.90 | 29.11 | 20.82 | 21.64 | 12.75 | 3.14 | 0.93 | 0.77 | 0.66 | 0.00 | 0.00 | 0.00 | 0.00 |
| 56 | 65.01 | 46.91 | 49.12 | 61.21 | 47.51 | 39.24 | 24.27 | 24.62 | 17.05 | 13.65 | 9.07 | 3.90 | 0.00 | 0.00 | 0.00 |
| Mean | 45.2 | 34.8 | 28.4 | 36.3 | 30.8 | 32.5 | 17.9 | 12.6 | 9.1 | 6.9 | 4.0 | 0.4 | 0.0 | 0.0 | 0.0 |
| Std.dev | 16.2 | 7.7 | 12.8 | 13.2 | 10.4 | 9.2 | 16.2 | 11.2 | 12.8 | 7.9 | 4.7 | 1.5 | 0.7 | 0.8 | 0.0 |

All wound measured on the line going from head to tail.
All wounds measured from the edge of the wound (between wound surface and normal skin).

TABLE 4
Relative Wound Areas

| 4 | Measuring day (Wound areas relative to Day 1) | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 25 | 1.00 | 0.28 | 0.26 | 0.28 | 0.30 | 0.32 | 0.31 | 0.27 | 0.12 | 0.11 | 0.09 | 0.02 | 0.02 | 0.00 | 0.00 |
| 26 | 1.00 | 1.06 | 0.89 | 1.05 | 0.95 | 2.46 | 2.44 | 2.52 | 0.77 | 0.41 | 0.57 | 0.26 | 0.28 | 0.00 | 0.34 |
| 27 | 1.00 | 0.76 | 0.73 | 0.52 | 0.29 | 0.75 | 0.90 | 0.89 | 0.96 | 0.71 | 0.85 | 0.50 | 0.50 | 0.22 | 0.26 |
| 28 | 1.00 | 0.72 | 0.64 | 1.02 | 1.06 | 1.08 | 0.95 | 0.68 | 0.29 | 0.23 | 0.23 | 0.09 | 0.10 | 0.01 | 0.01 |
| 29 | 1.00 | 0.63 | 0.59 | 1.12 | 1.58 | 1.08 | 1.01 | 0.91 | 0.55 | 0.24 | 0.22 | 0.12 | 0.08 | 0.08 | 0.10 |
| 30 | 1.00 | 0.84 | 0.77 | 1.03 | 0.90 | 1.28 | 1.44 | 1.48 | 1.52 | 3.18 | 1.02 | 0.72 | 0.82 | 0.75 | 0.63 |
| 31 | 1.00 | 1.02 | 1.06 | 1.03 | 1.28 | 1.03 | 0.93 | 0.96 | 0.24 | 0.20 | 0.19 | 0.17 | 0.02 | 0.00 | 0.00 |
| 32 | 1.00 | 0.52 | 0.36 | 0.58 | 0.64 | 0.76 | 0.25 | 0.28 | 0.02 | 0.08 | 0.07 | 0.04 | 0.02 | 0.00 | 0.06 |
| Mean | 1.0 | 0.7 | 0.6 | 0.8 | 0.8 | 1.0 | 0.9 | 0.8 | 0.4 | 0.4 | 0.3 | 0.2 | 0.1 | 0.0 | 0.1 |
| Std.dev. | 0.0 | 0.3 | 0.3 | 0.3 | 0.5 | 0.6 | 0.7 | 0.7 | 0.5 | 1.0 | 0.4 | 0.2 | 0.3 | 0.3 | 0.2 |
| 5 | Measuring day (Wound areas relative to Day 1) | | | | | | | | | | | | | | |
| Animal | 1 | 2 | 3 | 4 | 5 | 6 | 7 | 8 | 9 | 10 | 11 | 12 | 13 | 14 | 15 |
| 33 | 1.00 | 0.93 | 0.75 | 0.94 | 0.88 | 0.73 | 0.85 | 0.93 | 0.11 | 0.16 | 0.19 | 0.09 | 0.14 | 0.13 | 0.03 |
| 34 | 1.00 | 0.88 | 1.02 | 1.15 | 1.19 | 0.87 | 0.39 | 0.28 | 0.15 | 0.17 | 0.17 | 0.04 | 0.01 | 0.00 | 0.00 |
| 35 | 1.00 | 1.31 | 1.35 | 1.26 | 1.67 | 1.62 | 1.59 | 1.44 | 0.42 | 0.31 | 0.35 | 0.29 | 0.04 | 0.00 | 0.00 |
| 36 | 1.00 | 0.58 | 0.50 | 1.05 | 0.58 | 0.77 | 0.71 | 0.19 | 0.21 | 0.16 | 0.15 | 0.03 | 0.00 | 0.00 | 0.00 |
| 37 | 1.00 | 0.78 | 0.66 | 0.63 | 0.70 | 0.59 | 0.22 | 0.24 | 0.25 | 0.15 | 0.10 | 0.03 | 0.01 | 0.01 | 0.05 |
| 38 | 1.00 | 1.17 | 0.76 | 0.95 | 0.96 | 0.76 | 0.39 | 0.48 | 0.39 | 0.39 | 0.31 | 0.06 | 0.06 | 0.04 | 0.00 |
| 39 | 1.00 | 0.90 | 0.75 | 0.51 | 0.52 | 0.75 | 0.71 | 0.70 | 0.51 | 0.65 | 0.22 | 0.10 | 0.02 | 0.01 | 0.01 |
| 40 | 1.00 | 1.30 | 1.01 | 1.11 | 1.29 | 1.05 | 1.03 | 0.44 | 0.26 | 0.18 | 0.14 | 0.02 | 0.00 | 0.00 | 0.00 |
| Mean | 1.0 | 0.9 | 0.8 | 0.9 | 0.9 | 0.8 | 0.6 | 0.5 | 0.3 | 0.2 | 0.2 | 0.1 | 0.0 | 0.0 | 0.0 |
| Std.dev. | 0.0 | 0.3 | 0.3 | 0.3 | 0.4 | 0.3 | 0.4 | 0.4 | 0.1 | 0.2 | 0.1 | 0.1 | 0.0 | 0.0 | 0.0 |
| 6 | Measuring day (Wound areas relative to Day 1) | | | | | | | | | | | | | | |
| Animal | 1 | 2 | 3 | 4 | 5 | 6 | 7 | 8 | 9 | 10 | 11 | 12 | 13 | 14 | 15 |
| 41 | 1.00 | 0.56 | 0.59 | 0.66 | 0.60 | 0.18 | 0.14 | 0.11 | 0.00 | 0.00 | 0.00 | 0.00 | 0.00 | 0.00 | 0.00 |
| 42 | 1.00 | 0.79 | 0.70 | 0.72 | 0.70 | 0.57 | 0.29 | 0.15 | 0.09 | 0.09 | 0.10 | 0.01 | 0.00 | 0.00 | 0.00 |
| 43 | 1.00 | 0.68 | 0.61 | 0.64 | 0.69 | 0.33 | 0.12 | 0.10 | 0.08 | 0.08 | 0.06 | 0.01 | 0.00 | 0.00 | 0.00 |
| 44 | 1.00 | 0.73 | 0.69 | 1.34 | 2.39 | 0.90 | 1.23 | 1.37 | 1.37 | 1.45 | 0.77 | 0.44 | 0.20 | 0.21 | 0.10 |
| 45 | 1.00 | 0.97 | 1.02 | 0.93 | 0.99 | 0.99 | 0.93 | 0.46 | 0.46 | 0.45 | 0.15 | 0.05 | 0.02 | 0.00 | 0.00 |
| 46 | 1.00 | 1.22 | 0.93 | 0.86 | 0.94 | 1.01 | 1.28 | 1.31 | 1.39 | 1.57 | 1.41 | 1.45 | 0.46 | 0.51 | 0.54 |
| 47 | 1.00 | 1.68 | 1.59 | 1.03 | 1.54 | 0.77 | 0.71 | 0.35 | 0.35 | 0.38 | 0.19 | 0.01 | 0.00 | 0.00 | 0.00 |
| 48 | 1.00 | 1.27 | 1.12 | 1.11 | 1.14 | 1.10 | 0.33 | 0.33 | 0.33 | 0.17 | 0.08 | 0.00 | 0.00 | 0.00 | 0.00 |
| Mean | 1.0 | 0.9 | 0.9 | 0.9 | 1.0 | 0.7 | 0.5 | 0.4 | 0.3 | 0.3 | 0.2 | 0.1 | 0.0 | 0.0 | 0.0 |
| Std.dev. | 0.0 | 0.4 | 0.3 | 0.2 | 0.6 | 0.3 | 0.5 | 0.5 | 0.6 | 0.6 | 0.5 | 0.5 | 0.2 | 0.2 | 0.2 |
| 7 | Measuring day (Wound areas relative to Day 1) | | | | | | | | | | | | | | |
| Animal | 1 | 2 | 3 | 4 | 5 | 6 | 7 | 8 | 9 | 10 | 11 | 12 | 13 | 14 | 15 |
| 49 | 1.00 | 1.26 | 0.75 | 0.84 | 0.86 | 0.92 | 0.88 | 0.54 | 0.29 | 0.23 | 0.21 | 0.00 | 0.00 | 0.00 | 0.00 |
| 50 | 1.00 | 0.63 | 0.37 | 0.50 | 0.42 | 0.51 | 0.53 | 0.50 | 0.55 | 0.34 | 0.14 | 0.04 | 0.03 | 0.03 | 0.00 |
| 51 | 1.00 | 1.06 | 0.88 | 1.57 | 0.88 | 1.34 | 1.56 | 0.61 | 0.60 | 0.38 | 0.38 | 0.04 | 0.00 | 0.00 | 0.00 |
| 52 | 1.00 | 0.85 | 0.63 | 0.84 | 0.89 | 0.83 | 0.16 | 0.15 | 0.08 | 0.05 | 0.03 | 0.00 | 0.00 | 0.00 | 0.00 |
| 53 | 1.00 | 0.78 | 0.98 | 0.79 | 0.89 | 0.87 | 0.14 | 0.14 | 0.14 | 0.07 | 0.01 | 0.00 | 0.00 | 0.00 | 0.00 |
| 54 | 1.00 | 0.49 | 0.37 | 0.56 | 0.48 | 0.49 | 0.12 | 0.13 | 0.04 | 0.11 | 0.04 | 0.01 | 0.00 | 0.00 | 0.00 |
| 55 | 1.00 | 0.74 | 0.55 | 0.77 | 0.55 | 0.57 | 0.34 | 0.08 | 0.02 | 0.02 | 0.02 | 0.00 | 0.00 | 0.00 | 0.00 |
| 56 | 1.00 | 0.72 | 0.76 | 0.94 | 0.73 | 0.60 | 0.37 | 0.38 | 0.26 | 0.21 | 0.14 | 0.06 | 0.00 | 0.00 | 0.00 |
| Mean | 1.0 | 0.8 | 0.6 | 0.8 | 0.7 | 0.7 | 0.4 | 0.3 | 0.2 | 0.2 | 0.1 | 0.0 | 0.0 | 0.0 | 0.0 |
| Std.dev. | 0.0 | 0.2 | 0.2 | 0.3 | 0.2 | 0.3 | 0.5 | 0.2 | 0.2 | 0.1 | 0.1 | 0.0 | 0.0 | 0.0 | 0.0 |

TABLE 5
Animal weights

| | | Measuring day (g) | | | | |
|---|---|---|---|---|---|---|
| Group | Animal | 1 | 4 | 7 | 11 | 15 |
| 4 | 25 | 23.0 | 24.2 | 24.5 | 24.4 | 24.1 |
| | 26 | 21.4 | 24.1 | 23.9 | 22.9 | 23.2 |
| | 27 | 21.4 | 24.3 | 24.7 | 24.0 | 24.4 |
| | 28 | 18.9 | 21.5 | 23.4 | 23.1 | 22.3 |
| | 29 | 21.7 | 24.5 | 24.1 | 23.9 | 22.8 |
| | 30 | 19.3 | 20.1 | 21.9 | 22.0 | 21.8 |
| | 31 | 18.4 | 20.7 | 22.0 | 21.7 | 22.6 |
| | 32 | 20.0 | 21.5 | 22.2 | 21.5 | 21.4 |
| | Mean | 20.5 | 22.6 | 23.3 | 22.9 | 22.8 |
| | Std.dev. | 1.6 | 1.8 | 1.2 | 1.1 | 1.0 |
| 5 | 33 | 18.6 | 21.3 | 22.2 | 23.3 | 23.7 |
| | 34 | 18.3 | 19.4 | 21.1 | 21.1 | 23.6 |
| | 35 | 21.5 | 23.8 | 25.0 | 23.2 | 23.8 |
| | 36 | 20.5 | 23.6 | 23.8 | 24.0 | 24.3 |
| | 37 | 21.9 | 23.7 | 25.0 | 24.0 | 24.8 |
| | 38 | 21.6 | 22.9 | 23.4 | 24.0 | 24.3 |
| | 39 | 18.2 | 20.6 | 21.6 | 21.5 | 21.9 |
| | 40 | 20.1 | 22.9 | 23.9 | 23.7 | 23.6 |
| | Mean | 20.1 | 22.3 | 23.3 | 23.1 | 23.8 |
| | Std.dev. | 1.5 | 1.6 | 1.5 | 1.2 | 0.9 |
| 6 | 41 | 23.4 | 25.5 | 26.6 | 25.8 | 26.4 |
| | 42 | 21.0 | 22.4 | 22.0 | 21.4 | 21.3 |
| | 43 | 21.8 | 23.9 | 24.4 | 23.3 | 22.9 |
| | 44 | 21.0 | 22.9 | 23.6 | 23.0 | 22.7 |
| | 45 | 17.6 | 18.8 | 21.5 | 20.9 | 21.2 |
| | 46 | 17.4 | 18.2 | 19.9 | 20.1 | 21.5 |
| | 47 | 20.5 | 22.8 | 22.7 | 20.1 | 22.9 |
| | 48 | 19.2 | 19.4 | 20.2 | 22.9 | 23.5 |
| | Mean | 20.2 | 21.7 | 22.6 | 22.2 | 22.8 |
| | Std.dev. | 2.1 | 2.6 | 2.2 | 1.9 | 1.7 |
| 7 | 49 | 23.6 | 23.8 | 24.5 | 24.2 | 24.2 |
| | 50 | 19.9 | 22.5 | 22.7 | 22.0 | 23.1 |
| | 51 | 19.8 | 22.5 | 22.7 | 22.1 | 21.9 |
| | 52 | 19.9 | 21.9 | 22.3 | 21.9 | 23.1 |
| | 53 | 22.6 | 24.5 | 24.7 | 23.8 | 23.8 |
| | 54 | 21.9 | 24.2 | 23.6 | 24.3 | 25.0 |
| | 55 | 23.3 | 24.4 | 24.6 | 24.6 | 24.8 |
| | 56 | 19.4 | 20.6 | 21.0 | 21.2 | 21.8 |
| | Mean | 21.3 | 23.1 | 23.3 | 23.0 | 23.5 |
| | Std.dev. | 1.7 | 1.4 | 1.3 | 1.3 | 1.2 |

TABLE 6
Animal weights, relative to day 1

| | | Measuring day (Weights | | | | |
|---|---|---|---|---|---|---|
| | | relative to Day 1 in grams) | | | | |
| Group | Animal | 1 | 4 | 7 | 11 | 15 |
| 4 | 25 | 1.00 | 0.95 | 0.94 | 0.94 | 0.95 |
| | 26 | 1.00 | 0.89 | 0.90 | 0.93 | 0.92 |
| | 27 | 1.00 | 0.88 | 0.87 | 0.89 | 0.88 |
| | 28 | 1.00 | 0.88 | 0.81 | 0.82 | 0.85 |
| | 29 | 1.00 | 0.89 | 0.90 | 0.91 | 0.95 |
| | 30 | 1.00 | 0.96 | 0.88 | 0.88 | 0.89 |
| | 31 | 1.00 | 0.89 | 0.84 | 0.85 | 0.81 |
| | 32 | 1.00 | 0.93 | 0.90 | 0.93 | 0.93 |
| | Mean | 1.00 | 0.9 | 0.9 | 0.9 | 0.9 |
| | Std.dev. | 0.0 | 0.0 | 0.0 | 0.0 | 0.1 |
| 5 | 33 | 1.00 | 0.87 | 0.84 | 0.80 | 0.78 |
| | 34 | 1.00 | 0.94 | 0.87 | 0.87 | 0.78 |
| | 35 | 1.00 | 0.90 | 0.86 | 0.93 | 0.90 |
| | 36 | 1.00 | 0.87 | 0.86 | 0.85 | 0.84 |
| | 37 | 1.00 | 0.92 | 0.88 | 0.91 | 0.88 |
| | 38 | 1.00 | 0.94 | 0.92 | 0.90 | 0.89 |
| | 39 | 1.00 | 0.88 | 0.84 | 0.85 | 0.83 |
| | 40 | 1.00 | 0.88 | 0.84 | 0.85 | 0.85 |
| | Mean | 1.00 | 0.9 | 0.9 | 0.9 | 0.8 |
| | Std.dev. | 0.0 | 0.0 | 0.0 | 0.0 | 0.0 |
| 6 | 41 | 1.00 | 0.92 | 0.88 | 0.91 | 0.89 |
| | 42 | 1.00 | 0.94 | 0.95 | 0.98 | 0.99 |
| | 43 | 1.00 | 0.91 | 0.89 | 0.94 | 0.95 |
| | 44 | 1.00 | 0.92 | 0.89 | 0.91 | 0.93 |
| | 45 | 1.00 | 0.94 | 0.82 | 0.84 | 0.83 |
| | 46 | 1.00 | 0.96 | 0.87 | 0.87 | 0.81 |
| | 47 | 1.00 | 0.90 | 0.90 | 1.02 | 0.90 |
| | 48 | 1.00 | 0.99 | 0.95 | 0.84 | 0.82 |
| | Mean | 1.00 | 0.9 | 0.9 | 0.9 | 0.9 |
| | Std.dev. | 0.0 | 0.0 | 0.0 | 0.1 | 0.1 |
| 7 | 49 | 1.00 | 0.99 | 0.96 | 0.98 | 0.98 |
| | 50 | 1.00 | 0.88 | 0.88 | 0.90 | 0.86 |
| | 51 | 1.00 | 0.88 | 0.87 | 0.90 | 0.90 |
| | 52 | 1.00 | 0.91 | 0.89 | 0.91 | 0.86 |
| | 53 | 1.00 | 0.92 | 0.91 | 0.95 | 0.95 |
| | 54 | 1.00 | 0.90 | 0.93 | 0.90 | 0.88 |
| | 55 | 1.00 | 0.95 | 0.95 | 0.95 | 0.94 |
| | 56 | 1.00 | 0.94 | 0.92 | 0.92 | 0.89 |
| | Mean | 1.00 | 0.9 | 0.9 | 0.9 | 0.9 |
| | Std.dev. | 0.0 | 0.0 | 0.0 | 0.0 | 0.0 |

III. Accelerated wound healing in wild type mice

Introduction

Objective:

The purpose of the study was to test the pharmacological efficacy of Osteopontin on wound healing in normal wild type mice.

Summary:

Three groups of 8 C57BU6J mice were anesthetized, shaved and had an 8 mm wound made on their back with a 8 mm punch biopsy instrument at Day -2.

Groups were treated with Osteopontin (OPN) in salve as follows:

Group 1: Vehicle salve.

Group 2: 0.1% OPN salve.

Group 3: 1.0% OPN salve.

Treatment was repeated daily for 15 days. At the same time wounds were scored and measured from Day 1 to Day 15.

Treatment of wounds mice with 0.1% OPN salve appeared to have a significant positive effect on wound healing in the last part of the healing process.

Test Article

Description, Identification and Storage:

| | |
|---|---|
| Test article: | Osteopontin, topical application, 0.1% and 1.0%. |
| Vehicle: | Carrier salve |

Preparation:

Pipeline prepared stock and dose solution of Osteopontin in salve.

The stock solution was made at the beginning of the study. The dose solutions were stored in the refrigerator.

Formulation analysis:

Not performed Concentration and storage, stability and homogeneity:

Osteopontin powder was stored at room temperature.

Stock solution of Osteopontin was stored in refrigerator.

Test system

Species, strain and supplier

The study was performed in 24 female C57BU6J mice, 7-8 weeks, from M&B-Taconic.

Environment

The mice were housed 4 in a cage in standard Macrolon cages type 2.

Bedding was changed once a week in a laminar flow unit.

Temperature was 20° C. 24° C., and was controlled via the ambient ventilation system in the laboratory. Light cycle was 12-hour dark and 12-hour light (lights on 06.00).

Diet and Water

Diet was Altromin 1314 diet.

Water was UV-sterilized and water bottles were refilled when necessary during acclimatization and experiment.

Diet and water was administered ad libitum.

Animal Health and welfare

The animals had FELASA SPF-status and the housing and changing system was designed to assure that the SPF-status was preserved during the study.

Educated personnel under veterinary supervision handled the animals. Daily records and decisions were made concerning animal welfare.

Pre-experimental procedures

Acclimatization and health procedures

Animals were acclimatized for approximately 7 days.

Experimental procedures

Grouping

On Day -2 the mice were grouped according to the following set-up:

| Group | Animals | Treatment |
|---|---|---|
| 1 | 1-8 | Vehicle |
| 2 | 9-16 | Osteopontin 0.1% |
| 3 | 17-24 | Osteopontin 1.0% |

Analgesia

All mice were subjected to analgesia by administration of 5 mg/kg s.c.

Rimadryl 1 hour before wound procedures.

Introduction Wounds

Wounds were introduced on the mice on Day 1.

One hour after dosing of Rimadryl and approximately 15 minutes after surgical anesthesia was induced, one wound was induced on each mouse.

The wound was introduced on the dorsal skin of each mouse by the following procedure:

1. The back of the mouse was shaven in a 3×3 cm area

2. The 8 ml punch biopsy instrument was used to make a circular cut in the center area of the shaven area.

3. The skin piece was lifted a bit and carefully dissected free from the mouse.

Treatment

Treatment started on Day 1. The salves were then applied to all treatment groups as described in section 5.1.

Group 1 was treated with carrier salve, Group 2 was treated with 0.1% OPN in salve and Group 3 was treated with 1.0% OPN in salve. The salves were carefully applied topically to the wounds.

Treatment was repeated daily for 15 days.

Observations and Measurements

The wounds were scored and measured from Days 1-15.

The diameters of the wounds were measured longitudinally (on the line running from head to tail) and recorded each day.

The appearance of the wounds was scored and recorded each day according to the following system:

0: Completely healed

1: Small wound still detected.

2: No scab but still wound

3: No original scab but new scab (old scab fallen off but new scab created)

4: original scab still on wound.

Body weights were recorded on Days 1, 5, 8,12 and 15.

Termination

At termination the mice were euthanized.

Study overview

| Day | Date | |
|---|---|---|
| −7 | 03.03.2005 | Animals arrive at Pipeline Biotech |
| | | Acclimatization |
| 1 | 10.03.2005 | Introduction of wounds. |
| | | Treatment, measurements and scoring |
| 2 | | Treatment, measurements and scoring |
| 3 | | Treatment, measurements and scoring |
| 4 | | Treatment, measurements and scoring, body weights |
| 5 | | Treatment, measurements and scoring |
| 6 | | Treatment, measurements and scoring |
| 7 | | Treatment, measurements and scoring, body weights |
| 8 | | Treatment, measurements and scoring |
| 9 | | Treatment, measurements and scoring |
| 10 | | Treatment, measurements and scoring |
| 11 | | Treatment, measurements and scoring, body weights |
| 12 | | Treatment, measurements and scoring |
| 13 | | Treatment, measurements and scoring |
| 14 | | Treatment, measurements and scoring |
| 15 | 24.03.2004 | Treatment, measurements and scoring, body |
| | | weights |
| | | Termination |

Results

Wound Scores

Results are shown in Table 1 (appendix).

Data were analysed statistically by the Mann-Whitney paired Rank-sum test (Wilcoxon two sample test). Results in test groups were compared to Group 1. Results of the statistical analysis were as follows:

| | | | | | Day | Day | Day | Day | Day | Day |
|---|---|---|---|---|---|---|---|---|---|---|
| Group | Day 6 | Day 7 | Day 8 | Day 9 | 10 | 11 | 12 | 13 | 14 | 15 |
| 1 | — | — | — | — | — | — | — | — | — | — |
| 2 | ns | ns | ns | ns | ns | ns | ns | ns | ns | ns |
| 3 | ns | ns | ns | ns | ns | ns | ns | ns | ns | ns |

n.s.: non significant

Conclusions:

| | | |
|---|---|---|
| Group 1: | vehicle salve. | |
| Group 2: | 0.1% OPN salve. | Not different from Gr.1 |
| Group 3: | 1.0% OPN salve. | Not different from Gr.1 |

Wound Diameters

Results are shown in Table 2 (appendix).

Data were analysed statistically by paired F- and Student t-tests. Results in control and test groups were compared to Group 1. Results of the statistical analysis were as follows:

| | | | | | | | | | | Day | Day | Day | Day | Day | Day |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | 11 | 12 | 13 | 14 | 15 |
| 1 | — | — | — | — | — | — | — | — | — | — | — | — | — | — | — |
| 2 | ns | ns | <0.05 | ns | ns | ns | ns | ns | ns | <0.05 | <0.01 | <0.05 | <0.05 | ns | ns |
| 3 | ns | ns | ns | ns | ns | <0.05 | <0.05 | ns | ns | ns | ns | <0.05 | <0.05 | ns | ns |

n.s.: non significant
Results of AUC (Area Under Curve) statistical analysis of Wound diameters are as follows: (Results in control and test groups were compared to Group 1.)
Group 2: p < 0.05
Group 3: p < 0.05

Conclusions:

Group 1: vehicle salve.

Group 2: 0.1% OPN salve. Generally significantly lower diameters than Gr. 1

Group 3: 1.0% OPN salve. Generally significantly lower diameters than Gr.

Wound Areas

Results are shown in Table 3 (appendix).

Data were analysed statistically by paired F- and Student t-tests. Results in control and test groups were compared to Group 1. Results of the statistical analysis were as follows:

| | | | | | | | | | | Day | Day | Day | Day | Day | Day |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | 11 | 12 | 13 | 14 | 15 |
| 1 | — | — | — | — | — | — | — | — | — | — | — | — | — | — | — |
| 2 | ns | ns | ns | ns | ns | ns | ns | ns | ns | ns | <0.01 | <0.05 | ns | ns | ns |
| 3 | ns | ns | ns | ns | ns | ns | <0.05 | ns | ns | ns | ns | <0.05 | ns | ns | ns |

n.s.: non significant
Results of AUC (Area Under Curve) statistical analysis of Wound Areas are as follows: (Results in control and test groups were compared to Group 1.)
Group 2: <0.05
Group 3: Non significant.

Conclusions:

Group 1: vehicle salve.

Group 2: 0.1% OPN salve. Generally significantly lower areas than Gr. 1

Group 3: 1.0% OPN salve. Significantly lower wound areas on days 7 and 12.

Wound Areas relative to Day 1

Results are shown in Table 4 (appendix).

Data were analysed statistically by paired F- and Student t-tests. Results in control and test groups were compared to Group 1. Results of the statistical analysis were as follows:

| | | | | | | | | | | Day | Day | Day | Day | Day | Day |
|---|---|---|---|---|---|---|---|---|---|---|---|---|---|---|---|
| Group | Day 1 | Day 2 | Day 3 | Day 4 | Day 5 | Day 6 | Day 7 | Day 8 | Day 9 | 10 | 11 | 12 | 13 | 14 | 15 |
| 1 | — | — | — | — | — | — | — | — | — | — | — | — | — | — | — |
| 2 | — | ns | ns | ns | ns | ns | ns | ns | ns | <0.05 | <0.05 | <0.05 | ns | ns | ns |
| 3 | — | ns | ns | ns | ns | ns | ns | ns | ns | ns | ns | ns | ns | ns | ns |

n.s.: non significant
Results of AUC (Area Under Curve) statistical analysis of Relative Wound Areas are as follows: (Results in control and test groups were compared to Group 1.)
Group 2: Non significant.
Group 3: Non significant.

Conclusions:

| | |
|---|---|
| Group 1: | vehicle salve. |
| Group 2: | 0.1% OPN salve. Relative wound areas significantly |
| | lower than Gr. on days 10, 11 and 12 |
| Group 3: | 1.0% OPN salve. Not different from group 1. |

Body weights

Body weights of the animals are shown in Tables 5 and 6 (Appendix).

No differences in body weights were observed between the groups.

Conclusion

No difference was observed in wound scores between the three groups.

Both Group 2 (0.1% OPN) and Group 3 (1.0% OPN) had significantly lower wound diameters than Group 1 (Vehicle).

When wound areas were compared, only Group 2 appeared to have a general significant reduction in wound areas. Wound areas of Group 3 were only significantly smaller than Group 1 on days 7 and 12.

When wound areas were relativated to day 1, the relative wound areas of Group 2 was significantly smaller than Group 1 on days 10, 11 and 12. No significant differences were found between Groups 1 and 3.

Thus treatment of wounds on wild type mice with 0.1% OPN salve appeared to have a positive effect on wound healing in the final part of the healing process.

Archive

The final report as well as all raw data and results are kept in the archives of Pipeline Biotech ANS for a period of five (5) years from the end of the study.

TABLE 1
Wound scores:

| 1 | Scoring day | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 1 | 4 | 4 | 4 | 4 | 4 | 4 | 4 | 4 | 4 | 4 | 4 | 4 | 4 | 4 | 4 |
| 2 | 4 | 4 | 4 | 4 | 4 | 4 | 4 | 4 | 4 | 3 | 3 | 3 | 2 | 2 | 1 |
| 3 | 4 | 4 | 4 | 4 | 4 | 4 | 4 | 4 | 3 | 3 | 3 | 3 | 2 | 1 | 1 |
| 4 | 4 | 4 | 4 | 4 | 4 | 4 | 4 | 3 | 3 | 3 | 1 | 1 | 0 | 0 | 0 |
| 5 | 4 | 4 | 4 | 4 | 4 | 4 | 4 | 3 | 3 | 3 | 1 | 1 | 1 | 0 | 0 |
| 6 | 4 | 4 | 4 | 4 | 4 | 4 | 4 | 4 | 3 | 3 | 1 | 1 | 1 | 0 | 0 |
| 7 | 4 | 4 | 4 | 4 | 4 | 4 | 3 | 3 | 3 | 3 | 1 | 1 | 1 | 0 | 0 |
| 8 | 4 | 4 | 4 | 4 | 4 | 4 | 4 | 4 | 3 | 3 | 1 | 1 | 1 | 0 | 0 |
| Mean | 4.0 | 4.0 | 4.0 | 4.0 | 4.0 | 4.0 | 3.9 | 3.6 | 3.3 | 3.1 | 1.9 | 1.9 | 1.5 | 0.9 | 0.8 |
| Std.dev | — | — | — | — | — | — | 0.4 | 0.5 | 0.5 | 0.4 | 1.2 | 1.2 | 1.2 | 1.5 | 1.4 |
| 2 | Scoring day | | | | | | | | | | | | | | |
| Animal | 1 | 2 | 3 | 4 | 5 | 6 | 7 | 8 | 9 | 10 | 11 | 12 | 13 | 14 | 15 |
| 9 | 4 | 4 | 4 | 4 | 4 | 4 | 4 | 4 | 3 | 3 | 3 | 1 | 0 | 0 | 0 |
| 10 | 4 | 4 | 4 | 4 | 4 | 4 | 4 | 3 | 3 | 3 | 2 | 1 | 0 | 0 | 0 |
| 11 | 4 | 4 | 4 | 4 | 4 | 4 | 4 | 3 | 3 | 3 | 2 | 3 | 1 | 0 | 0 |
| 12 | 4 | 4 | 4 | 4 | 4 | 4 | 3 | 3 | 3 | 3 | 2 | 3 | 3 | 0 | 0 |
| 13 | 4 | 4 | 4 | 4 | 4 | 4 | 4 | 4 | 3 | 3 | 2 | 1 | 0 | 0 | 0 |
| 14 | 4 | 4 | 4 | 4 | 4 | 4 | 4 | 4 | 3 | 3 | 2 | 1 | 1 | 0 | 0 |
| 15 | 4 | 4 | 4 | 4 | 4 | 4 | 3 | 3 | 0 | 0 | 0 | 0 | 0 | 0 | 0 |
| 16 | 4 | 4 | 4 | 4 | 4 | 3 | 3 | 3 | 3 | 3 | 3 | 3 | 0 | 0.5 | 0 |
| Mean | 4.0 | 4.0 | 4.0 | 4.0 | 4.0 | 3.9 | 3.6 | 3.4 | 2.6 | 2.6 | 2.0 | 1.6 | 0.6 | 0.1 | 0 |
| Std.dev | — | — | — | — | — | 0.4 | 0.5 | 0.5 | 1.1 | 1.1 | 0.9 | 1.2 | 1.1 | 0.2 | — |
| 3 | Scoring day | | | | | | | | | | | | | | |
| Animal | 1 | 2 | 3 | 4 | 5 | 6 | 7 | 8 | 9 | 10 | 11 | 12 | 13 | 14 | 15 |
| 17 | 4 | 4 | 4 | 4 | 4 | 4 | 3 | 3 | 3 | 2 | 2 | 1 | 0 | 0 | 0 |
| 18 | 4 | 4 | 4 | 4 | 4 | 4 | 4 | 4 | 3 | 3 | 3 | 3 | 1 | 1 | 0 |
| 19 | 4 | 4 | 4 | 4 | 4 | 4 | 4 | 4 | 4 | 3 | 3 | 2 | 2 | 2 | 1 |
| 20 | 4 | 4 | 4 | 4 | 4 | 4 | 4 | 4 | 3 | 3 | 3 | 1 | 0 | 0 | 0 |
| 21 | 4 | 4 | 4 | 4 | 4 | 4 | 4 | 3 | 3 | 3 | 2 | 1 | 0 | 0 | 0 |
| 22 | 4 | 4 | 4 | 4 | 4 | 4 | 2 | 3 | 0 | 0 | 0 | 3 | 0 | 0 | 0 |
| 23 | 4 | 4 | 4 | 4 | 4 | 4 | 4 | 4 | 3 | 3 | 3 | 3 | 2 | 0 | 0 |
| 24 | 4 | 4 | 4 | 4 | 4 | 4 | 4 | 4 | 4 | 4 | 4 | 2 | 2 | 1 | 0 |
| Mean | 4.0 | 4.0 | 4.0 | 4.0 | 4.0 | 4.0 | 3.6 | 3.6 | 2.9 | 2.6 | 2.5 | 2.0 | 0.9 | 0.5 | 0.1 |
| Std.dev | — | — | — | — | — | — | 0.7 | 0.5 | 1.2 | 1.2 | 1.2 | 0.9 | 1.0 | 0.8 | 0.4 |

Scoring:
0: Completely healed
1: Small wound still detected.
2: No scab but still wound
3: No original scab but new scab (old scab fallen off but new scab created)
4: original scab still on wound.

TABLE 2
Wound Diameters

| 1 | Measuring day (mm) | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 1 | 6.72 | 5.53 | 5.16 | 5.08 | 4.98 | 6.30 | 6.12 | 6.36 | 5.91 | 5.50 | 5.62 | 4.78 | 4.63 | 4.60 | 4.11 |
| 2 | 10.52 | 9.47 | 10.08 | 10.07 | 9.83 | 9.80 | 8.82 | 8.71 | 8.56 | 8.33 | 5.24 | 3.85 | 2.26 | 2.31 | 1.60 |
| 3 | 6.77 | 5.16 | 5.64 | 5.26 | 5.32 | 6.40 | 5.65 | 5.23 | 5.10 | 4.06 | 3.56 | 3.53 | 1.86 | 1.91 | 2.08 |
| 4 | 6.52 | 8.52 | 9.62 | 10.12 | 9.99 | 8.52 | 8.06 | 3.84 | 3.97 | 3.98 | 3.62 | 2.85 | 0 | 0 | 0 |
| 5 | 6.04 | 7.26 | 7.35 | 6.34 | 6.58 | 8.08 | 6.22 | 3.92 | 3.89 | 3.15 | 2.88 | 0.79 | 0.53 | 0 | 0 |
| 6 | 8.97 | 7.91 | 7.41 | 9.04 | 9.09 | 10.55 | 8.11 | 8.41 | 3.69 | 3.39 | 3.32 | 1.86 | 0.67 | 0 | 0 |
| 7 | 7.89 | 5.81 | 5.92 | 4.57 | 4.69 | 4.54 | 4.44 | 3.36 | 3.01 | 2.41 | 2.09 | 1.38 | 0.66 | 0 | 0 |
| 8 | 6.65 | 7.26 | 7.86 | 6.55 | 6.41 | 7.03 | 6.30 | 6.29 | 3.52 | 2.92 | 2.50 | 1.52 | 0.89 | 0 | 0 |
| Mean | 7.5 | 7.1 | 7.4 | 7.1 | 7.1 | 7.7 | 6.7 | 5.8 | 4.7 | 4.2 | 3.6 | 2.6 | 1.4 | 1.1 | 1.0 |
| Std.dev | 1.5 | 1.5 | 1.8 | 2.3 | 2.2 | 2.0 | 1.5 | 2.1 | 1.8 | 1.9 | 1.2 | 1.4 | 1.5 | 1.7 | 1.5 |
| 2 | Measuring day (mm) | | | | | | | | | | | | | | |
| Animal | 1 | 2 | 3 | 4 | 5 | 6 | 7 | 8 | 9 | 10 | 11 | 12 | 13 | 14 | 15 |
| 9 | 4.90 | 5.89 | 4.45 | 5.95 | 6.03 | 3.92 | 3.70 | 4.07 | 2.45 | 2.74 | 2.26 | 0.83 | 0 | 0 | 0 |
| 10 | 8.11 | 6.91 | 5.82 | 6.69 | 6.07 | 6.39 | 5.40 | 4.26 | 2.50 | 2.27 | 2.27 | 0.81 | 0 | 0 | 0 |
| 11 | 8.35 | 6.78 | 6.44 | 6.15 | 5.35 | 6.49 | 5.31 | 5.25 | 2.80 | 2.13 | 2.13 | 1.10 | 0.78 | 0 | 0 |
| 12 | 4.12 | 3.52 | 3.03 | 3.46 | 4.04 | 3.90 | 3.87 | 2.60 | 2.61 | 2.21 | 2.00 | 0.93 | 1.11 | 0 | 0 |
| 13 | 6.68 | 7.78 | 8.55 | 8.61 | 8.75 | 8.89 | 7.34 | 6.80 | 3.56 | 2.88 | 0.66 | 0.66 | 0 | 0 | 0 |
| 14 | 6.79 | 6.28 | 5.77 | 7.17 | 7.08 | 6.66 | 5.68 | 6.05 | 6.05 | 2.56 | 2.31 | 0.83 | 0.61 | 0 | 0 |
| 15 | 4.04 | 6.69 | 5.71 | 5.25 | 5.41 | 6.07 | 4.89 | 3.80 | 0 | 0 | 0 | 0.61 | 0 | 0 | 0 |
| 16 | 8.25 | 7.78 | 6.80 | 5.74 | 5.59 | 7.06 | 6.88 | 6.29 | 5.19 | 3.31 | 1.59 | 0 | 0.78 | 0.85 | 0 |
| Mean | 6.4 | 6.5 | 5.8 | 6.1 | 6.0 | 6.2 | 5.4 | 4.9 | 3.1 | 2.3 | 1.7 | 0.7 | 0.4 | 0.1 | 0.0 |
| Std.dev | 1.8 | 1.4 | 1.6 | 1.5 | 1.4 | 1.6 | 1.3 | 1.4 | 1.9 | 1.0 | 0.9 | 0.3 | 0.5 | 0.3 | 0.0 |
| 3 | Measuring day (mm) | | | | | | | | | | | | | | |
| Animal | 1 | 2 | 3 | 4 | 5 | 6 | 7 | 8 | 9 | 10 | 11 | 12 | 13 | 14 | 15 |
| 17 | 6.38 | 4.73 | 5.33 | 4.99 | 4.96 | 5.64 | 5.47 | 5.52 | 5.46 | 2.91 | 2.22 | 0.91 | 0 | 0 | 0 |
| 18 | 6.20 | 7.60 | 6.70 | 6.46 | 6.51 | 6.51 | 6.35 | 4.19 | 4.39 | 3.74 | 3.03 | 0.99 | 0.84 | 0 | 0 |
| 19 | 5.58 | 9.13 | 9.42 | 9.01 | 9.25 | 8.77 | 7.38 | 7.04 | 4.37 | 4.85 | 4.10 | 1.94 | 0.41 | 1.20 | 1.44 |
| 20 | 8.83 | 6.42 | 6.50 | 5.75 | 5.29 | 5.98 | 6.29 | 6.38 | 4.19 | 3.99 | 2.13 | 1.07 | 0 | 0 | 0 |
| 21 | 7.03 | 6.73 | 6.08 | 4.00 | 4.19 | 4.42 | 3.87 | 2.68 | 2.46 | 1.92 | 1.48 | 0.49 | 0 | 0 | 0 |
| 22 | 4.46 | 5.12 | 4.55 | 4.89 | 4.66 | 5.32 | 2.89 | 2.44 | 0 | 0 | 0 | 0 | 0 | 0 | 0 |
| 23 | 7.41 | 8.12 | 7.31 | 6.46 | 5.85 | 7.09 | 5.03 | 5.23 | 5.20 | 3.66 | 2.69 | 2.35 | 1.14 | 0 | 0 |
| 24 | 4.78 | 7.34 | 5.50 | 5.88 | 5.84 | 5.73 | 4.51 | 5.06 | 4.57 | 4.78 | 4.69 | 1.42 | 1.02 | 1.00 | 0 |
| Mean | 6.3 | 6.9 | 6.4 | 5.9 | 5.8 | 6.2 | 5.2 | 4.8 | 3.8 | 3.2 | 2.5 | 1.1 | 0.4 | 0.3 | 0.2 |
| Std.dev | 1.4 | 1.5 | 1.5 | 1.5 | 1.6 | 1.3 | 1.5 | 1.6 | 1.8 | 1.6 | 1.5 | 0.8 | 0.5 | 0.5 | 0.5 |

All wound measured on the line going from head to tail.
All wounds measured from the edge of the wound (between wound surface and normal skin).

TABLE 3
Wound Areas

| 1 | Measuring day (mm²) | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 1 | 35.45 | 24.01 | 20.90 | 20.26 | 19.47 | 31.16 | 29.40 | 31.75 | 27.42 | 23.75 | 24.79 | 17.94 | 16.83 | 16.61 | 13.26 |
| 2 | 86.88 | 70.40 | 79.76 | 79.60 | 75.85 | 75.39 | 61.07 | 59.55 | 57.52 | 54.47 | 21.55 | 11.64 | 4.01 | 4.19 | 2.01 |
| 3 | 35.98 | 20.90 | 24.97 | 21.72 | 22.22 | 32.15 | 25.06 | 21.47 | 20.42 | 12.94 | 9.95 | 9.78 | 2.72 | 2.86 | 3.40 |
| 4 | 33.37 | 56.98 | 72.65 | 80.40 | 78.34 | 56.98 | 51.00 | 11.58 | 12.37 | 12.43 | 10.29 | 6.38 | 0.00 | 0.00 | 0.00 |
| 5 | 28.64 | 41.38 | 42.41 | 31.55 | 33.99 | 51.25 | 30.37 | 12.06 | 11.88 | 7.79 | 6.51 | 0.49 | 0.22 | 0.00 | 0.00 |
| 6 | 63.16 | 49.12 | 43.10 | 64.15 | 64.86 | 87.37 | 51.63 | 55.52 | 10.69 | 9.02 | 8.65 | 2.72 | 0.35 | 0.00 | 0.00 |
| 7 | 48.87 | 26.50 | 27.51 | 16.39 | 17.27 | 16.18 | 15.48 | 8.86 | 7.11 | 4.56 | 3.43 | 1.49 | 0.34 | 0.00 | 0.00 |
| 8 | 34.71 | 41.38 | 48.50 | 33.68 | 32.25 | 38.80 | 31.16 | 31.06 | 9.73 | 6.69 | 4.91 | 1.81 | 0.62 | 0.00 | 0.00 |
| Mean | 44.3 | 39.7 | 42.8 | 39.9 | 39.7 | 46.0 | 35.4 | 26.1 | 17.4 | 14.0 | 10.2 | 5.2 | 1.6 | 1.0 | 0.7 |
| Std.dev | 19.9 | 17.3 | 21.7 | 26.9 | 25.8 | 24.0 | 15.7 | 19.6 | 16.7 | 16.4 | 7.8 | 6.2 | 5.7 | 5.8 | 4.6 |
| 2 | Measuring day (mm²) | | | | | | | | | | | | | | |
| Animal | 1 | 2 | 3 | 4 | 5 | 6 | 7 | 8 | 9 | 10 | 11 | 12 | 13 | 14 | 15 |
| 9 | 18.85 | 27.23 | 15.54 | 27.79 | 28.54 | 12.06 | 10.75 | 13.00 | 4.71 | 5.89 | 4.01 | 0.54 | 0.00 | 0.00 | 0.00 |
| 10 | 51.63 | 37.48 | 26.59 | 35.13 | 28.92 | 32.05 | 22.89 | 14.25 | 4.91 | 4.05 | 4.05 | 0.52 | 0.00 | 0.00 | 0.00 |
| 11 | 54.73 | 36.09 | 32.56 | 29.69 | 22.47 | 33.06 | 22.13 | 21.64 | 6.15 | 3.56 | 3.56 | 0.95 | 0.48 | 0.00 | 0.00 |
| 12 | 13.32 | 9.73 | 7.21 | 9.40 | 12.81 | 11.94 | 11.76 | 5.31 | 5.35 | 3.83 | 3.14 | 0.68 | 0.97 | 0.00 | 0.00 |
| 13 | 35.03 | 47.51 | 57.39 | 58.19 | 60.10 | 62.04 | 42.29 | 36.30 | 9.95 | 6.51 | 0.34 | 0.34 | 0.00 | 0.00 | 0.00 |
| 14 | 36.19 | 30.96 | 26.13 | 40.36 | 39.35 | 34.82 | 25.33 | 28.73 | 28.73 | 5.14 | 4.19 | 0.54 | 0.29 | 0.00 | 0.00 |
| 15 | 12.81 | 35.13 | 25.59 | 21.64 | 22.98 | 28.92 | 18.77 | 11.34 | 0.00 | 0.00 | 0.00 | 0.29 | 0.00 | 0.00 | 0.00 |
| 16 | 53.43 | 47.51 | 36.30 | 25.86 | 24.53 | 39.13 | 37.16 | 31.06 | 21.14 | 8.60 | 1.98 | 0.00 | 0.48 | 0.57 | 0.00 |
| Mean | 32.2 | 32.7 | 26.6 | 29.5 | 28.6 | 29.9 | 22.8 | 18.8 | 7.8 | 4.0 | 2.1 | 0.4 | 0.1 | 0.0 | 0.0 |
| Std.dev | 17.8 | 12.1 | 14.9 | 14.3 | 14.3 | 15.9 | 11.1 | 11.0 | 9.7 | 2.5 | 1.7 | 0.3 | 0.4 | 0.2 | 0.0 |
| 3 | Measuring day (mm²) | | | | | | | | | | | | | | |
| Animal | 1 | 2 | 3 | 4 | 5 | 6 | 7 | 8 | 9 | 10 | 11 | 12 | 13 | 14 | 15 |
| 17 | 31.95 | 17.56 | 22.30 | 19.55 | 19.31 | 24.97 | 23.49 | 23.92 | 23.40 | 6.65 | 3.87 | 0.65 | 0.00 | 0.00 | 0.00 |
| 18 | 30.18 | 45.34 | 35.24 | 32.76 | 33.27 | 33.27 | 31.65 | 13.78 | 15.13 | 10.98 | 7.21 | 0.77 | 0.55 | 0.00 | 0.00 |
| 19 | 24.44 | 65.44 | 69.66 | 63.73 | 67.17 | 60.38 | 42.75 | 38.91 | 14.99 | 18.47 | 13.20 | 2.95 | 0.13 | 1.13 | 1.63 |
| 20 | 61.21 | 32.35 | 33.17 | 25.95 | 21.97 | 28.07 | 31.06 | 31.95 | 13.78 | 12.50 | 3.56 | 0.90 | 0.00 | 0.00 | 0.00 |
| 21 | 38.80 | 35.55 | 29.02 | 12.56 | 13.78 | 15.34 | 11.76 | 5.64 | 4.75 | 2.89 | 1.72 | 0.19 | 0.00 | 0.00 | 0.00 |
| 22 | 15.61 | 20.58 | 16.25 | 18.77 | 17.05 | 22.22 | 6.56 | 4.67 | 0.00 | 0.00 | 0.00 | 0.00 | 0.00 | 0.00 | 0.00 |
| 23 | 43.10 | 51.76 | 41.95 | 32.76 | 26.86 | 39.46 | 19.86 | 21.47 | 21.23 | 10.52 | 5.68 | 4.34 | 1.02 | 0.00 | 0.00 |
| 24 | 17.94 | 42.29 | 23.75 | 27.14 | 26.77 | 25.77 | 15.97 | 20.10 | 16.39 | 17.94 | 17.27 | 1.58 | 0.82 | 0.79 | 0.00 |
| Mean | 31.5 | 37.4 | 32.4 | 27.6 | 26.6 | 30.0 | 21.4 | 18.2 | 11.5 | 8.2 | 5.1 | 1.0 | 0.1 | 0.1 | 0.0 |
| Std.dev | 14.8 | 15.9 | 16.6 | 15.6 | 16.9 | 13.8 | 11.9 | 11.9 | 7.8 | 6.6 | 5.9 | 1.5 | 0.4 | 0.5 | 0.6 |

All wound measured on the line going from head to tail.
All wounds measured from the edge of the wound (between wound surface and normal skin).

TABLE 4
Relative Wound Areas

| 1 | Measuring day (Wound areas relative to Day 1) | | | | | | | | | | | | | | |
|---|---|---|---|---|---|---|---|---|---|---|---|---|---|---|---|
| Animal | 1 | 2 | 3 | 4 | 5 | 6 | 7 | 8 | 9 | 10 | 11 | 12 | 13 | 14 | 15 |
| 1 | 1.00 | 0.68 | 0.59 | 0.57 | 0.55 | 0.88 | 0.83 | 0.90 | 0.77 | 0.67 | 0.70 | 0.51 | 0.47 | 0.47 | 0.37 |
| 2 | 1.00 | 0.81 | 0.92 | 0.92 | 0.87 | 0.87 | 0.70 | 0.69 | 0.66 | 0.63 | 0.25 | 0.13 | 0.05 | 0.05 | 0.02 |
| 3 | 1.00 | 0.58 | 0.69 | 0.60 | 0.62 | 0.89 | 0.70 | 0.60 | 0.57 | 0.36 | 0.28 | 0.27 | 0.08 | 0.08 | 0.09 |
| 4 | 1.00 | 1.71 | 2.18 | 2.41 | 2.35 | 1.71 | 1.53 | 0.35 | 0.37 | 0.37 | 0.31 | 0.19 | 0.00 | 0.00 | 0.00 |
| 5 | 1.00 | 1.44 | 1.48 | 1.10 | 1.19 | 1.79 | 1.06 | 0.42 | 0.41 | 0.27 | 0.23 | 0.02 | 0.01 | 0.00 | 0.00 |
| 6 | 1.00 | 0.78 | 0.68 | 1.02 | 1.03 | 1.38 | 0.82 | 0.88 | 0.17 | 0.14 | 0.14 | 0.04 | 0.01 | 0.00 | 0.00 |
| 7 | 1.00 | 0.54 | 0.56 | 0.34 | 0.35 | 0.33 | 0.32 | 0.18 | 0.15 | 0.09 | 0.07 | 0.03 | 0.01 | 0.00 | 0.00 |
| 8 | 1.00 | 1.19 | 1.40 | 0.97 | 0.93 | 1.12 | 0.90 | 0.89 | 0.28 | 0.19 | 0.14 | 0.05 | 0.02 | 0.00 | 0.00 |
| Mean | 1.0 | 0.9 | 1.0 | 0.9 | 0.9 | 1.0 | 0.8 | 0.6 | 0.4 | 0.3 | 0.2 | 0.1 | 0.0 | 0.0 | 0.0 |
| Std.dev. | 0.0 | 0.4 | 0.6 | 0.6 | 0.6 | 0.5 | 0.3 | 0.3 | 0.2 | 0.2 | 0.2 | 0.2 | 0.2 | 0.2 | 0.1 |
| 2 | Measuring day (Wound areas relative to Day 1) | | | | | | | | | | | | | | |
| Animal | 1 | 2 | 3 | 4 | 5 | 6 | 7 | 8 | 9 | 10 | 11 | 12 | 13 | 14 | 15 |
| 9 | 1.00 | 1.44 | 0.82 | 1.47 | 1.51 | 0.64 | 0.57 | 0.69 | 0.25 | 0.31 | 0.21 | 0.03 | 0.00 | 0.00 | 0.00 |
| 10 | 1.00 | 0.73 | 0.51 | 0.68 | 0.56 | 0.62 | 0.44 | 0.28 | 0.10 | 0.08 | 0.08 | 0.01 | 0.00 | 0.00 | 0.00 |
| 11 | 1.00 | 0.66 | 0.59 | 0.54 | 0.41 | 0.60 | 0.40 | 0.40 | 0.11 | 0.07 | 0.07 | 0.02 | 0.01 | 0.00 | 0.00 |
| 12 | 1.00 | 0.73 | 0.54 | 0.71 | 0.96 | 0.90 | 0.88 | 0.40 | 0.40 | 0.29 | 0.24 | 0.05 | 0.07 | 0.00 | 0.00 |
| 13 | 1.00 | 1.36 | 1.64 | 1.66 | 1.72 | 1.77 | 1.21 | 1.04 | 0.28 | 0.19 | 0.01 | 0.01 | 0.00 | 0.00 | 0.00 |
| 14 | 1.00 | 0.86 | 0.72 | 1.12 | 1.09 | 0.96 | 0.70 | 0.79 | 0.79 | 0.14 | 0.12 | 0.01 | 0.01 | 0.00 | 0.00 |
| 15 | 1.00 | 2.74 | 2.00 | 1.69 | 1.79 | 2.26 | 1.47 | 0.88 | 0.00 | 0.00 | 0.00 | 0.02 | 0.00 | 0.00 | 0.00 |
| 16 | 1.00 | 0.89 | 0.68 | 0.48 | 0.46 | 0.73 | 0.70 | 0.58 | 0.40 | 0.16 | 0.04 | 0.00 | 0.01 | 0.01 | 0.00 |
| Mean | 1.0 | 1.0 | 0.8 | 0.9 | 0.9 | 0.9 | 0.7 | 0.6 | 0.2 | 0.1 | 0.1 | 0.0 | 0.0 | 0.0 | 0.0 |
| Std.dev. | 0.0 | 0.7 | 0.6 | 0.5 | 0.6 | 0.6 | 0.4 | 0.3 | 0.2 | 0.1 | 0.1 | 0.0 | 0.0 | 0.0 | 0.0 |
| 3 | Measuring day (Wound areas relative to Day 1) | | | | | | | | | | | | | | |
| Animal | 1 | 2 | 3 | 4 | 5 | 6 | 7 | 8 | 9 | 10 | 11 | 12 | 13 | 14 | 15 |
| 17 | 1.00 | 0.55 | 0.70 | 0.61 | 0.60 | 0.78 | 0.74 | 0.75 | 0.73 | 0.21 | 0.12 | 0.02 | 0.00 | 0.00 | 0.00 |
| 18 | 1.00 | 1.50 | 1.17 | 1.09 | 1.10 | 1.10 | 1.05 | 0.46 | 0.50 | 0.36 | 0.24 | 0.03 | 0.02 | 0.00 | 0.00 |
| 19 | 1.00 | 2.68 | 2.85 | 2.61 | 2.75 | 2.47 | 1.75 | 1.59 | 0.61 | 0.76 | 0.54 | 0.12 | 0.01 | 0.05 | 0.07 |
| 20 | 1.00 | 0.53 | 0.54 | 0.42 | 0.36 | 0.46 | 0.51 | 0.52 | 0.23 | 0.20 | 0.06 | 0.01 | 0.00 | 0.00 | 0.00 |
| 21 | 1.00 | 0.92 | 0.75 | 0.32 | 0.36 | 0.40 | 0.30 | 0.15 | 0.12 | 0.07 | 0.04 | 0.00 | 0.00 | 0.00 | 0.00 |
| 22 | 1.00 | 1.32 | 1.04 | 1.20 | 1.09 | 1.42 | 0.42 | 0.30 | 0.00 | 0.00 | 0.00 | 0.00 | 0.00 | 0.00 | 0.00 |
| 23 | 1.00 | 1.20 | 0.97 | 0.76 | 0.62 | 0.92 | 0.46 | 0.50 | 0.49 | 0.24 | 0.13 | 0.10 | 0.02 | 0.00 | 0.00 |
| 24 | 1.00 | 2.36 | 1.32 | 1.51 | 1.49 | 1.44 | 0.89 | 1.12 | 0.91 | 1.00 | 0.96 | 0.09 | 0.05 | 0.04 | 0.00 |
| Mean | 1.0 | 1.2 | 1.0 | 0.9 | 0.8 | 1.0 | 0.7 | 0.6 | 0.4 | 0.3 | 0.2 | 0.0 | 0.0 | 0.0 | 0.0 |
| Std.dev. | 0.0 | 0.8 | 0.7 | 0.7 | 0.8 | 0.7 | 0.5 | 0.5 | 0.3 | 0.3 | 0.3 | 0.0 | 0.0 | 0.0 | 0.0 |

TABLE 5
Animal weights

| | | Measuring day (g) | | | | |
|---|---|---|---|---|---|---|
| Group | Animal | 1 | 4 | 7 | 11 | 15 |
| 1 | 1 | 21.2 | 22.2 | 22.1 | 21.6 | 21.0 |
| | 2 | 22.0 | 22.2 | 23.1 | 21.2 | 22.0 |
| | 3 | 20.7 | 21.3 | 21.0 | 20.9 | 22.3 |
| | 4 | 20.5 | 20.3 | 23.0 | 20.8 | 21.3 |
| | 5 | 19.0 | 18.4 | 19.8 | 19.8 | 19.6 |
| | 6 | 18.4 | 17.8 | 18.2 | 17.4 | 18.8 |
| | 7 | 19.6 | 20.0 | 20.5 | 20.7 | 20.1 |
| | 8 | 21.1 | 21.1 | 22.5 | 21.8 | 22.9 |
| | Mean | 20.3 | 20.4 | 21.3 | 20.5 | 21.0 |
| | Std.dev. | 1.2 | 1.6 | 1.7 | 1.4 | 1.4 |
| 2 | 9 | 21.3 | 21.5 | 21.9 | 21.1 | 20.8 |
| | 10 | 23.3 | 24.2 | 24.9 | 23.7 | 23.6 |
| | 11 | 20.4 | 21.8 | 23.3 | 22.3 | 22.3 |
| | 12 | 20.7 | 20.9 | 21.2 | 21.5 | 21.5 |
| | 13 | 20.9 | 22.0 | 22.6 | 21.9 | 23.4 |
| | 14 | 23.1 | 24.2 | 24.7 | 24.2 | 24.2 |
| | 15 | 19.7 | 20.0 | 20.8 | 21.0 | 22.1 |
| | 16 | 20.2 | 20.6 | 19.7 | 20.8 | 22.3 |
| | Mean | 21.2 | 21.9 | 22.4 | 22.1 | 22.5 |
| | Std.dev. | 1.3 | 1.6 | 1.8 | 1.3 | 1.1 |
| 3 | 17 | 22.7 | 23.9 | 23.9 | 22.8 | 23.0 |
| | 18 | 21.0 | 21.4 | 21.4 | 20.5 | 21.1 |
| | 19 | 22.3 | 22.1 | 22.8 | 22.6 | 23.5 |
| | 20 | 21.8 | 22.2 | 23.9 | 22.2 | 22.6 |
| | 21 | 19.6 | 20.5 | 20.8 | 21.0 | 20.4 |
| | 22 | 22.3 | 22.4 | 23.7 | 22.9 | 22.4 |
| | 23 | 22.8 | 23.7 | 24.0 | 24.0 | 23.2 |
| | 24 | 22.3 | 22.6 | 22.6 | 22.8 | 21.9 |
| | Mean | 21.9 | 22.4 | 22.9 | 22.4 | 22.3 |
| | Std.dev. | 1.1 | 1.1 | 1.2 | 1.1 | 1.1 |

TABLE 6
Animal weights, relative to day 1

| | | Measuring day (Weights | | | | |
|---|---|---|---|---|---|---|
| | | relative to Day 1 in grams) | | | | |
| Group | Animal | 1 | 4 | 7 | 11 | 15 |
| 1 | 1 | 1.00 | 0.95 | 0.96 | 0.98 | 1.01 |
| | 2 | 1.00 | 0.99 | 0.95 | 1.04 | 1.00 |
| | 3 | 1.00 | 0.97 | 0.99 | 0.99 | 0.93 |
| | 4 | 1.00 | 1.01 | 0.89 | 0.99 | 0.96 |
| | 5 | 1.00 | 1.03 | 0.96 | 0.96 | 0.97 |
| | 6 | 1.00 | 1.03 | 1.01 | 1.06 | 0.98 |
| | 7 | 1.00 | 0.98 | 0.96 | 0.95 | 0.98 |
| | 8 | 1.00 | 1.00 | 0.94 | 0.97 | 0.92 |
| | Mean | 1.0 | 1.0 | 1.0 | 1.0 | 1.0 |
| | Std.dev. | 0.0 | 0.0 | 0.0 | 0.0 | 0.0 |
| 2 | 9 | 1.00 | 0.99 | 0.97 | 1.01 | 1.02 |
| | 10 | 1.00 | 0.96 | 0.94 | 0.98 | 0.99 |
| | 11 | 1.00 | 0.94 | 0.88 | 0.91 | 0.91 |
| | 12 | 1.00 | 0.99 | 0.98 | 0.96 | 0.96 |
| | 13 | 1.00 | 0.95 | 0.92 | 0.95 | 0.89 |
| | 14 | 1.00 | 0.95 | 0.94 | 0.95 | 0.95 |
| | 15 | 1.00 | 0.99 | 0.95 | 0.94 | 0.89 |
| | 16 | 1.00 | 0.98 | 1.03 | 0.97 | 0.91 |
| | Mean | 1.0 | 1.0 | 0.9 | 1.0 | 0.9 |
| | Std.dev. | 0.0 | 0.0 | 0.0 | 0.0 | 0.0 |
| 3 | 17 | 1.00 | 0.95 | 0.95 | 1.00 | 0.99 |
| | 18 | 1.00 | 0.98 | 0.98 | 1.02 | 1.00 |
| | 19 | 1.00 | 1.01 | 0.98 | 0.99 | 0.95 |
| | 20 | 1.00 | 0.98 | 0.91 | 0.98 | 0.96 |
| | 21 | 1.00 | 0.96 | 0.94 | 0.93 | 0.96 |
| | 22 | 1.00 | 1.00 | 0.94 | 0.97 | 1.00 |
| | 23 | 1.00 | 0.96 | 0.95 | 0.95 | 0.98 |
| | 24 | 1.00 | 0.99 | 0.99 | 0.98 | 1.02 |
| | Mean | 1.0 | 1.0 | 1.0 | 1.0 | 1.0 |
| | Std.dev. | 0.0 | 0.0 | 0.0 | 0.0 | 0.0 |

### MAIN CONCLUSION

The experiments were carried out on healthy and diabetic mice and also on infected, healthy animals.

In order to impact the healing in an acute wound in a normally perfused tissue, the effect of the examined test substance must be rather strong. The osteopontin test has demonstrated an effect also on these models, in particular at the end of the wound healing process. There seems to be some minor differences as to the osteopontin concentrations to be used, but this may be due to the structure of the various wound models. Bovine osteopontin is seen to have an effect on diabetic animals as well as on an infected wound which heals in spite of the induced bacteria. This substantiates a wound healing effect of bovine osteopontin. That an effect can also be seen on perfectly healthy animals supports this assumption. Therefore main conclusion would be that bovine osteopontin has an effect on the healing of acute wounds. Hence, there is also reason to believe that this effect applies to chronic non-healing wounds as well.

1. A wound healing topical formulation comprising bovine osteopontin and an adjuvant.

2. A formulation of claim 1, where the formulation is an ointment, a liquid, a powder or a plaster.

3. An ointment of claim 2, where the level of osteopontin is 0.01% to 10%.

4. An ointment of claim 3, where the level of osteopontin is 0.1% to 5%.

5. An ointment of claim 4, where the level of osteopontin is 0.1% to 2%.

6. An ointment of claim 5, where the level of osteopontin is 0.1% to 1.5%.

7. An ointment of claim 6, where the level of osteopontin is 0.5% to 1%.

8. Use of bovine osteopontin for the preparation of a topical drug formulation for improved wound healing.

9. Use of bovine osteopontin for the preparation of a topical drug formulation for improved healing of infected or inflamed wounds.

10. Use of bovine osteopontin for the preparation of a topical drug formulation for improved healing of diabetic wounds.

11. A method for improved wound healing comprising administering to a patient in need thereof an effective amount of bovine osteopontin.
